(19) United States
(12) Patent Application Publication
(10) Pub. No.: US 2008/0057131 A1
(43) Pub. Date: Mar. 6, 2008

(54) Method of modifying the release profile of sustained release compositions

(75) Inventors: James R. Dasch, Needham, MA (US); M. Gary I. Riley, Boston, MA (US); Paul A. Burke, Oxnard, CA (US); Susan A. Steitz-Abadi, Barrington, RI (US); Stephen E. Zale, Hopkinton, MA (US)

Correspondence Address: COVINGTON & BURLING, LLP;ATTN: PATENT DOCKETING, 1201 PENNSYLVANIA AVENUE, N.W., WASHINGTON, DC, 20004-2401, US

(73) Assignee: Alkermes Controlled Therapeutics, Inc., Cambridge, MA (US)

(21) Appl. No.: 11/826,694

(22) Filed: Jul. 17, 2007

Related U.S. Application Data
(63) Continuation of application No. 10/681,571, filed on Oct. 8, 2003.
(60) Provisional application No. 60/419,430, filed on Oct. 17, 2002.

(51) Int. Cl. A61K 38/02; A61K 9/00; A61P 43/00

(52) U.S. Cl. 424/499; 514/2

(57) ABSTRACT

The present invention relates to a method for the sustained release in vivo of a biologically active labile agent comprising administering to a subject in need of treatment an effective amount of a sustained release composition comprising a biocompatible polymer having the biologically active labile agent incorporated therein, and a corticosteroid wherein the labile is released for a period of at least about two weeks. It is understood that the corticosteroid is present in an amount sufficient to modify the release profile of the biologically active labile agent from the sustained release composition. Pharmaceutical compositions suitable for use in the method of the invention are also disclosed.

### BACKGROUND OF THE INVENTION

Many illnesses or conditions require administration of a constant or sustained level of a medicament or biologically active agent to provide the most effective prophylactic or therapeutic. This may be accomplished through a multiple dosing regimen or by employing a system that releases the medicament in a sustained fashion.

Attempts to sustain medication levels include the use of biodegradable materials, such as polymeric matrices, containing the medicament. The use of these matrices, for example, in the form of microparticles or microcarriers, provides sustained release of medicaments by utilizing the inherent biodegradability of the polymer. The ability to provide a sustained level of medicament can result in improved patient compliance.

However, these sustained release devices can exhibit high release of active agent initially, which can result in an undesirable increase in the levels of biologically active agent and minimal release of agent thereafter. Further, due to the high solution concentration of medicament within and localized around these sustained release devices, the medicament can be altered thereby increasing immunogenicity in vivo and interfering with the desired release profile for the medicament. This is particularly common when the medicament is a labile agent such as a protein or peptide.

In addition, parenteral delivery of a sustained release device to a patient can sometimes trigger a local foreign body response (FBR) at the site of delivery. This local response can affect the release kinetics and bioavailability of the medicaments contained in the microparticles particularly when the medicament is a labile agent such as a protein or peptide.

Therefore, a need exists to exert additional control over the release profile of sustained release compositions thereby providing an improved composition.

### SUMMARY OF THE INVENTION

The present invention is based upon the unexpected discovery that the release profile of a biologically active labile agent from a sustained release composition comprising a biocompatible polymer and the biologically active labile agent incorporated therein can be modified when a corticosteroid is co-administered. Modification of the release profile results in increased bioavailability of the encapsulated biologically active labile agent.

In addition, a sustained release composition comprising a biocompatible polymer, a biologically active labile agent and a corticosteroid, can also modulate an immune response by the host to the sustained release composition. The response can result from the encapsulated biologically active labile agent, can be a general foreign body response resulting from the composition or a combination thereof.

It has been found that the increase in bioavailability is most notable in sustained release formulations with a targeted release period of biologically active labile agent of at least about two weeks or longer, for example, at least about three weeks or longer, such as at least about four weeks or longer. That is, the improvement in the release profile of the administered sustained release composition is most notable at or about 2 weeks post administration. Typically, an extension of duration of release from about 25%-35% has been obtained for formulations targeted for a one month or longer release.

Accordingly, the present invention relates to a method for the sustained release in vivo of a biologically active labile agent comprising administering to a subject in need of treatment an effective amount of a sustained release composition comprising a biocompatible polymer having the biologically active labile agent incorporated therein, and a corticosteroid. It is preferred that the labile agent is released for a period of at least about two weeks, such as at least about three weeks, for example, at least about 4 weeks. It is understood that the corticosteroid is present in an amount sufficient to modify the release profile of the biologically active labile agent from the sustained release composition.

In one embodiment, the corticosteroid can be co-incorporated into the sustained release composition comprising the biocompatible polymer and the biologically active labile agent incorporated therein.

In another embodiment, the corticosteroid can be separately incorporated into a second biocompatible polymer. The biocompatible polymer can be the same or different from the first biocompatible polymer which has the biologically active labile agent incorporated therein.

In yet another embodiment, the corticosteroid can be present in an unencapsulated state but commingled with the sustained release composition. For example, the corticosteroid can be solubilized in the vehicle used to deliver the sustained release composition. Alternatively, the corticosteroid can be present as a solid suspended in an appropriate vehicle. Further, the corticosteroid can be present as a powder which is commingled with the sustained release composition.

The invention described herein also relates to pharmaceutical compositions suitable for use in the invention. In one embodiment, the pharmaceutical composition comprises a sustained release composition comprising a biocompatible polymer having an effective amount of a biologically active labile agent incorporated therein, and a corticosteroid. It is preferred that the labile agent in released for a period of at least about two weeks. For example, release of the agent can be for a period of at least about three weeks, such as at least about four weeks. It is understood that the corticosteroid is present in an amount sufficient to modify the release profile of the biologically active labile agent from the sustained release composition or to modulate an immune response by the host to the sustained release composition.

In one embodiment, the corticosteroid can be co-incorporated into the sustained release composition comprising the biocompatible polymer and the biologically active labile agent incorporated therein.

In another embodiment, the pharmaceutical composition comprises the sustained release composition comprising a first biocompatible polymer having incorporated therein an effective amount of a biologically active labile agent and a second biocompatible polymer having incorporated therein a corticosteroid. It is understood that the corticosteroid modifies the release profile of the biologically active labile agent from the first polymer and/or modulates an immune response by the host to the sustained release composition. In a particular embodiment, the first and second polymers are the same type of polymer. In another embodiment, the first and second polymers are different.

In yet another embodiment, the corticosteroid can be present in the pharmaceutical composition in an unencapsulated state. For example, the corticosteroid can be commingled with the sustained release composition. In one embodiment, the corticosteroid can be solubilized in the vehicle used to deliver the pharmaceutical composition. Alternatively, the corticosteroid can be present as a solid suspended in an appropriate vehicle useful for delivering the pharmaceutical composition. Further, the corticosteroid can be present as a powder which is commingled with the sustained release composition.

Without being bound by a particular theory, it is believed that at least in part the effects of the corticosteroid on the bioavailability of the labile agent can be related to a reduction in the amount of inflammatory cellular reaction which can occur in the area of administration of the sustained release composition. The inflammatory reaction can be in response to the presence of a foreign body, the biologically active agent, the polymer or a combination thereof. For example, a polymer used to encapsulate the biologically active labile agent can elicit an inflammatory reaction. This response, although clinically insignificant, is well characterized as a foreign body response, and can be realized with most foreign materials. It has been appreciated herein that such an inflammatory reaction can decrease the overall efficacy of the sustained release composition. The decrease can require that the clinical microparticle be larger, which can create administration and injection site difficulties.

The corticosteroid, in addition to enhancing the bioavailability of the biologically active labile agent, can also modulate the ability of the host animal to mount an immune response to the encapsulated biological active labile substance. For example, administration of a corticosteroid with a biologically active labile agent can dampen the potential for antibody formation to the biologically active labile agent. The corticosteroid can also alter expression and/or presence of pro-inflammatory cytokines at the site of administration of the biologically active labile agent which can improve the release profile.

### BRIEF DESCRIPTION OF THE DRAWINGS

The foregoing and other objects, features and advantages of the invention will be apparent from the following more particular description of preferred embodiments of the invention, as illustrated in the accompanying drawings.

FIG. 1 is a graph of serum EPO levels (mU/mL) in rats administered EPO-containing microparticles, EPO-containing microparticles admixed with hydrocortisone acetate (5 mg), or EPO-containing microparticles admixed with triamcinolone diacetate (1 mg or 5 mg) over time (days).

FIG. 2 is a graph of hematocrit values (%) in rats administered EPO-containing microparticles, EPO-containing microparticles admixed with hydrocortisone acetate (5 mg), or EPO-containing microparticles admixed with triamcinolone diacetate (1 mg or 5 mg) over time (days).

FIG. 3A is a graph of serum EPO levels (mU/ml) in rats administered microparticles containing EPO co-encapsulated with hydrocortisone at various levels and EPO-containing microparticles admixed with hydrocortisone acetate (5 mg).

FIG. 3B is a graph of hematocrit values (%) in rats administered microparticles containing EPO co-encapsulated with hydrocortisone at various levels (0.25, 2.0, 14%) and EPO-containing microparticles admixed with hydrocortisone acetate (5 mg) versus time (days).

FIG. 4 is a graph of serum EPO levels (mU/mL) in rats administered EPO-containing microparticles in combination with 100 mg of placebo microparticles, 5 mg of triamcinolone acetonide and 100 mg of placebo microparticles admixed or 100 mg of 20% w/w hydrocortisone-containing microparticles over time (days).

FIG. 5 is a graph of hematocrit values (%) in rats administered 100 mg of placebo microparticles, 5 mg of triamcinolone acetonide and 100 mg of placebo microparticles admixed or 100 mg of 20% w/w hydrocortisone-containing microparticles over time (days).

FIG. 6A is a graph of the incidence of antibodies to EPO (titer) detected in the serum of rats administered a total of 10,000 Units of EPO in combination with a total of 100 mg of placebo microparticles, 5 mg of triamcinolone acetonide and 100 mg of placebo microparticles admixed or 100 mg of 20% w/w hydrocortisone-containing microparticles at day 12 after administration.

FIG. 6B is a graph of the incidence of antibodies to EPO (titer) detected in the serum of rats administered a total of 10,000 Units of EPO in combination with a total of 100 mg of placebo microparticles, 5 mg of triamcinolone acetonide and 100 mg of placebo microparticles admixed or 100 mg of 20% w/w hydrocortisone-containing microparticles at day 19 after administration.

FIG. 6C is a graph of the incidence of antibodies to EPO (titer) detected in the serum of rats administered a total of 10,000 Units of EPO in combination with a total of 100 mg of placebo microparticles, 5 mg of triamcinolone acetonide and 100 mg of placebo microparticles admixed or 100 mg of 20% w/w hydrocortisone-containing microparticles at day 33 after administration.

FIG. 7A is a graph of serum EPO levels (mU/mL) in rats administered EPO-containing microparticles admixed with placebo microparticles, dexamethasone-containing microparticles, budesonide containing microparticles and triamcinolone acetonide-containing microparticles versus time (days).

FIG. 7B is a graph of hematocrit values (%) in rats administered EPO-containing microparticles admixed with placebo microparticles, dexamethasone-containing microparticles, budesonide containing microparticles and triamcinolone acetonide-containing microparticles versus time (days).

FIG. 8A is a graph of serum EPO levels (mU/mL) in rats administered EPO-containing microparticles admixed with placebo microparticles, triamcinolone acetonide-containing microparticles (5, 10, 20 mg), and budesonide-containing microparticles (25 and 50 mg) as well as microparticles having EPO and triamcinolone acetonide co-encapsulated over time (days).

FIG. 8B is a graph of hematocrit values (%) in rats administered EPO-containing microparticles admixed with placebo microparticles, triamcinolone acetonide-containing microparticles (5, 10, 20 mg) and microparticles having EPO and triamcinolone acetonide co-encapsulated (Top Panel), and placebo microparticles and budesonide-containing microparticles (25, 50 mg) (Bottom Panel) over time (days).

FIG. 9 is a graph of serum hFSH levels (mIU/mL) in rats administered hFSH-containing microparticles in combination with a total of 75 mg of placebo microparticles, 10 mg of 2% w/w triamcinolone acetonide-containing microparticles, or mg of 2% w/w hydrocortisone-containing microparticles over time (days).

FIG. 10 is a graph of serum hFSH levels (mIU/mL) in rats administered hFSH-containing microparticles in combination with a total of 100 mg of placebo microparticles or 10 mg of 2% triamcinolone acetonide-containing microparticles with 90 mg of placebo microparticles.

FIG. 11 is a graph of serum insulin levels (mU/mL) in rats administered 60 mg of insulin-containing microparticles plus 75 mg of placebo, 10 mg of 2% w/w triamcinolone acetonide-containing microparticles or 15 mg of 2% w/w hydrocortisone-containing microparticles over time (days).

FIG. 12 is a histogram of osteopontin mRNA expression levels (copy numbers/50 ng cDNA) in rats administered 60 mg of insulin-containing microparticles plus 75 mg of placebo, 10 mg of 2% w/w triamcinolone acetonide-containing microparticles, IS mg of 2% w/w hydrocortisone-containing microparticles at day 14 after administration.

FIG. 13 is a graph of serum insulin levels (mU/mL) in rats administered 60 mg of insulin-containing microparticles plus 25 mg of placebo, 10 mg of 2% w/w triamcinolone acetonide-containing microparticles or 15 mg of 2% w/w hydrocortisone-containing microparticles over time (days).

FIG. 14 is a histogram of osteopontin mRNA expression levels (copy numbers/50 ng cDNA) in rats administered 60 mg of insulin-containing microparticles plus 25 mg of placebo microparticles, 10 mg of 2% w/w triamcinolone-containing microparticles, 15 mg of 2% w/w hydrocortisone-containing microparticles at days 7 and 35 after administration.

FIG. 15 is a graph of serum exendin-4 levels (pg/mL) in rats administered 120 mg of exendin-containing microparticles plus 30 mg of placebo microparticles or 10 mg of 2% triamcinolone acetonide-containing microparticles versus time in days.

FIG. 16 is a graph of serum exendin-4 levels (pg/mL) in rats administered 40 mg of exendin-containing microparticles plus 30 mg of placebo microparticles or 10 mg of 2% triamcinolone acetonide-containing microparticles versus time in days.

### DETAILED DESCRIPTION OF THE INVENTION

A description of preferred embodiments of the invention follows.

The present invention relates to a method for the sustained release in vivo of a biologically active labile agent comprising administering to a subject in need of treatment an effective amount of a sustained release composition comprising a biocompatible polymer having the biologically active labile agent incorporated therein, and a corticosteroid. It is preferred that said agent is released for a period of at least about two weeks, such as at least about three weeks, for example at least about four weeks. The corticosteroid as such, is present in an amount sufficient to modify the release profile of the biologically active labile agent from the sustained release composition, to modulate an immune response by a host to the biologically active agent or a combination thereof.

In one embodiment, the corticosteroid can be co-incorporated into the sustained release composition comprising the biocompatible polymer and the biologically active labile agent incorporated therein.

In another embodiment, the corticosteroid can be separately incorporated into a second biocompatible polymer. The second biocompatible polymer can be the same or different from the first biocompatible polymer which has the biologically active labile agent incorporated therein.

In yet another embodiment, the corticosteroid can be present in an unencapsulated state but commingled with the sustained release composition. For example, the corticosteroid can be solubilized in the vehicle used to deliver the sustained release composition. Alternatively, the corticosteroid can be present as a solid suspended in an appropriate vehicle. Further, the corticosteroid can be present as a powder which is commingled with the sustained release composition.

“Patient” as that term is used herein refers to a human.

The term “sustained release composition” as defined herein, comprises a biocompatible polymer having incorporated therein at least one biologically active labile agent. It is preferred that the labile agent is released for a period of at lest about two weeks, such as at least about three weeks, such as at least about four weeks. Suitable biocompatible polymers, can be either biodegradable or non-biodegradable polymers or blends or copolymers thereof, as described herein.

Typically, the sustained release composition can contain from about 0.01% (w/w) to about 50% (w/w) of the biologically active labile agent (dry weight of composition). The amount of agent used will vary depending upon the desired effect of the agent, the planned release levels, and the time span over which the agent will be released. A preferred range of agent loading is between about 0.1% (w/w) to about 30% (w/w) agent. A more preferred range of agent loading is between about 0.5% (w/w) to about 20% (w/w) agent.

The sustained release compositions of this invention can be formed into many shapes such as a film, a pellet, a rod, a filament, a cylinder, a disc, a wafer or a microparticle. A microparticle is preferred. A “microparticle” as defined herein, comprises a polymer component having a diameter of less than about one millimeter and having a biologically active labile agent dispersed therein. A microparticle can have a spherical (e.g., a microsphere), non-spherical or irregular shape. Typically, the microparticle will be of a size suitable for injection. A preferred size range for microparticles is from about one to about 180 microns in diameter.

As defined herein, a “sustained release of biologically active labile agent” is a release of the agent from a sustained release composition. The release occurs over a period which is longer than that period during which a therapeutically significant amount of the biologically active labile agent, would be available following direct administration of a solution of the biologically active labile agent. It is preferred that a sustained release be a release of biologically active labile agent which occurs over a period of at least about two weeks or more, for example, about three weeks or more such as about four weeks or more. The sustained release composition can therefore be prepared to have a targeted delivery of about two weeks or more, such as about three weeks or more, for example, 4 weeks or more. A sustained release of biologically active labile agent, from a sustained release composition can be a continuous or a discontinuous release, with relatively constant or varying rates of release. The continuity of release and level of release can be affected by the type of polymer composition used (e.g., monomer ratios, molecular weight, and varying combinations of polymers), agent loading, and/or selection of excipients to produce the desired effect.

As used herein, “sufficient corticosteroid to modify the release profile of the biologically active labile agent from the biocompatible polymer” means that amount of corticosteroid which modifies the release profile of the biologically active labile agent from the biocompatible polymer in comparison to the release which occurs when the sustained release composition does not include a corticosteroid.

“Modifies the release profile” as that term is used herein refers to increased bioavailability of the biologically active agent of the sustained release composition.

“Bioavailability” as that term is used herein refers to the amount of therapeutic that reaches the general circulation. That is, the calculated Area Under the Curve (AUC) for the release profile of a particular labile during the time period starting at 2 days post administration (also referred to as the post burst period) and ending at a predetermined time point. As is understood in the art, the release profile is generated by graphing the serum levels of a biologically active agent in a subject (Y-axis) at predetermined time point (X-axis). Bioavailability is often referred to in terms of % Bioavailability, which is the bioavailablity achieved for a particular polypeptide following administration of a sustained release composition divided by the bioavailability achieved for a particular polypeptide following administration of the same dose of drug intravenously multiplied by 100.

“Increased bioavailability” as that term is used herein refers to an increase in the bioavailability of a biologically active labile agent from a sustained release compositions when coadministered with a corticosteroid in comparison to the administration in the absence of corticosteroid over a time period beginning at two days post administration and ending at the targeted timepoint for the particular formulation.

A modification of the release profile can be confirmed by appropriate pharmacokinetic monitoring of the patient's serum for the presence of the biologically active labile agent. For example, specific antibody-based testing (e.g., ELISA and IRMA), as is well known in the art, can be used to determine the concentration of certain biologically active labile agents in the patient's serum. An example of such testing is described herein for erythropoietin, follicle stimulating hormone, and insulin.

Pharmacodynamic monitoring of the patient to monitor the therapeutic effects of the agent upon the patient can be used to confirm retention of the biologically activity of the released agent. For example, determination of the patient's hematocrit in response to administration of erythropoietin, as described herein. Methods of monitoring pharmacodynamic effects can be selected based upon the biologically active labile agent being administered using widely available techniques.

As used herein, “sufficient corticosteroid to modulate an immune response by a host to the biologically active labile agent” means that amount of corticosteroid that modifies an immune response to a biologically active labile agent in a host which occurs when the sustained release composition containing the biologically active labile agent does not include the corticosteroid. Modulation of an immune response by the host can be detected in a number of ways, for example, by detecting antibodies to the biologically active labile agent, for example, as described herein or any other methods known to one of skill in the art.

As used herein, a “therapeutically effective amount”, “prophylactically effective amount” or “diagnostically effective amount” is the amount of the sustained release composition needed to elicit the desired biological response following administration.

Corticosteroids, as defined herein, refers to steroidal anti-inflammatory agents also referred to as glucocorticoids.

21-Acetoxypregnenolone, Alclometasone, Algestone, Amcinonide, Beclomethasone, Betamethasone, Budesonide, Chloroprednisone, Clobetasol, Clobetasone, Clocortolone, Cloprednol, Corticosterone, Cortisone, Cortivazol, Deflazacort, Desonide, Desoximetasone, Dexamethasone, Disflorasone, Diflucortolone, Difluprednate, Enoxolone, Fluazacort, Flucloronide, Flumethasone, Flunisolide, Flucinolone Acetonide, Fluocinonide, Fluocortin Butyl, Flucortolone, Fluorometholone, Fluperolone Acetate, Fluprednidene Acetate, Fluprednisolone, Flurandrenolide, Fluticasone Propionate, Formocortal, Halcinonide, Halobetasol Propionate, Halometasone, Halopredone Acetate, Hydrocortamate, Hydrocortisone, Loteprednol Etabonate, Mazipredone, Medrysone, Meprednisone, Methylprednisolone, Mometasone Furoate, Paramethasone, Prednicarbate, Prednisolone, Prednisolone 25-Diethylamino-acetate, Prednisolone Sodium Phosphate, Prednisone, Prednival, Prednylidene, Rimexolone, Tixocortol, Triamcinolone (all forms), for example, Triamcinolone Acetonide, Triamcinolone Acetonide 21-oic acid methyl ester, Triamcinolone Benetonide, Triamcinolone Hexacetonide, Triamcinolone Diacetate, pharmaceutically acceptable mixtures thereof and salts thereof and any other derivative and analog thereof.

As used herein, the term “a” or “an” refers to one or more.

The polymers of the invention are biocompatible. Suitable biocompatible polymers, can be either biodegradable or non-biodegradable polymers or blends or copolymers thereof, as described herein.

Suitable biocompatible polymers, can be either biodegradable or non-biodegradable polymers or blends or copolymers thereof, as described herein. A polymer is biocompatible if the polymer and any degradation products of the polymer are non-toxic to the recipient and also possess no significant deleterious or untoward effects on the recipient's body, such as an immunological reaction at the injection site.

“Biodegradable”, as defined herein, means the composition will degrade or erode in vivo to form smaller chemical species. Degradation can result, for example, by enzymatic, chemical and physical processes. Suitable biocompatible, biodegradable polymers include, for example, poly(lactides), poly(glycolides), poly(lactide-co-glycolides), poly(lactic acid)s, poly(glycolic acid)s, polycarbonates, polyesteramides, polyanydrides, poly(amino acids), polyorthoesters, poly(dioxanone)s, poly(alkylene alkylate)s, copolymers or polyethylene glycol and polyorthoester, biodegradable polyurethane, blends thereof, and copolymers thereof.

Suitable biocompatible, non-biodegradable polymers include non-biodegradable polymers selected from the group consisting of polyacrylates, polymers of ethylene-vinyl acetates and other acyl substituted cellulose acetates, non-degradable polyurethanes, polystyrenes, polyvinylchloride, polyvinyl flouride, poly(vinyl imidazole), chlorosulphonate polyolefins, polyethylene oxide, blends thereof, and copolymers thereof.

Acceptable molecular weights for polymers used in this invention can be determined by a person of ordinary skill in the art taking into consideration factors such as the desired polymer degradation rate, physical properties such as mechanical strength, and rate of dissolution of polymer in solvent. Typically, an acceptable range of molecular weight is of about 2,000 Daltons to about 2,000,000 Daltons.

In a particular embodiment, the polymer is biodegradable polymer or copolymer. In a more preferred embodiment, the polymer is a poly(lactide-co-glycolide)(hereinafter “PLG”). The PLG can have a lactide:glycolide ratio, for example, of about 10:90, 25:75, 50:50, 75:25 or 90:10 and a molecular weight of about 5,000 Daltons to about 70,000 Daltons.

The term “biologically active labile agent,” as used herein, refers to a protein or peptide, or its pharmaceutically acceptable salt, which when released in vivo, possesses the desired biological activity, for example therapeutic, diagnostic and/or prophylactic properties in vivo. It is understood that the term includes stabilized biologically active labile agents as described herein.

Examples of suitable biologically active labile agents include proteins such as immunoglobulins, antibodies, cytokines (e.g., lymphokines, monokines, chemokines), interleukins, interferons, erythropoietin, nucleases, tumor necrosis factor, colony stimulating factors, insulin, enzymes (e.g., superoxide dismutase, plasminogen activator, etc.), tumor suppressors, blood proteins, hormones and hormone analogs (e.g., follicle stimulating hormone, growth hormone, adrenocorticotropic hormone, and luteinizing hormone releasing hormone (LHRH)), vaccines (e.g., tumoral, bacterial and viral antigens), antigens, blood coagulation factors; growth factors; and peptides such as protein inhibitors, protein antagonists, and protein agonists for example exendin-4, GLP-1, gastrin, GRH, antibacterial peptide such as defensin, enkephalins, bradykinins and calcitonin.

In one embodiment, the biologically active labile agent is stabilized. The biologically active labile agent can be stabilized against degradation, loss of potency and/or loss of biological activity, all of which can occur during formation of the sustained release composition having the biologically active labile agent dispersed therein, and/or prior to and during in vivo release of the biologically active labile agent. In one embodiment, stabilization can result in a decrease in the solubility of the biologically active labile agent, the consequence of which is a reduction in the initial release of biologically active labile agent, in particular, when release is from a sustained release composition. In addition, the period of release of the biologically active labile agent can be prolonged.

Stabilization of the biologically active labile agent can be accomplished, for example, by the use of a stabilizing agent or a specific combination of stabilizing agents. The stabilizing agent can be present in the mixture. “Stabilizing agent”, as that term is used herein, is any agent which binds or interacts in a covalent or non-covalent manner or is included with the biologically active labile agent. Stabilizing agents suitable for use in the invention are described in U.S. Pat. Nos. 5,716,644, 5,674,534, 5,654,010, 5,667,808, and 5,711,968, and published PCT Application WO96/40074 to Burke et al., having a publication date of Dec. 19, 1996 and U.S. Pat. No. 6,265,389 to Burke, issued on Jul. 24, 2001 and U.S. Application No. 60/419,388 entitled, “Microencapsulation and Sustained Release of Biologically Active Polypeptide” by Henry R. Costantino et al. filed on Oct. 17, 2002, the entire teachings of all of which are incorporated herein by reference.

For example, a metal cation can be complexed with the biologically active labile agent, or the biologically active labile agent can be complexed with a polycationic complexing agent such as protamine, albumin, spermidine and spermine, or associated with a “salting-out” salt. In addition, a specific combination of stabilizing agents and/or excipients may be needed to optimize stabilization of the biologically active labile agent.

Suitable metal cations include any metal cation capable of complexing with the biologically active labile agent. A metal cation-stabilized biologically active labile agent, as defined herein, comprises a biologically active labile agent and at least one type of metal cation wherein the cation is not significantly oxidizing to the biologically active labile agent. In a particular embodiment, the metal cation is multivalent, for example, having a valency of +2 or more. It is preferred that the metal cation be complexed to the biologically active labile agent.

Suitable stabilizing metal cations include biocompatible metal cations. A metal cation is biocompatible if the cation is non-toxic to the recipient, in the quantities used, and also presents no significant deleterious or untoward effects on the recipient's body, such as a significant immunological reaction at the injection site. The suitability of metal cations for stabilizing biologically active labile agents and the ratio of metal cation to biologically active labile agent needed can be determined by one of ordinary skill in the art by performing a variety of stability indicating techniques such as polyacrylamide gel electrophoresis, isoelectric focusing, reverse phase chromatography, and HPLC analysis on particles of metal cation-stabilized biologically active labile agents prior to and following particle size reduction and/or encapsulation. The molar ratio of metal cation to biologically active labile agent is typically between about 1:2 and about 100:1, preferably between about 2:1 and about 12:1.

Examples of stabilizing metal cations include, but are not limited to, K⁺, Zn⁺², Mg⁺² and Ca⁺². Stabilizing metal cations also include cations of transition metals, such as Cu⁺². Combinations of metal cations can also be employed.

The biologically active labile agent can also be stabilized with at least one polycationic complexing agent. Suitable polycationic complexing agents include, but are not limited to, protamine, spermine, spermidine and albumin. The suitability of polycationic complexing agents for stabilizing biologically active labile agents can be determined by one of ordinary skill in the art in the manner described above for stabilization with a metal cation. An equal weight ratio of polycationic complexing agent to biologically active labile agent is suitable.

Further, excipients can be added to maintain the potency of the biologically active labile agent over the duration of release and modify polymer degradation. The excipients. Suitable excipients include, for example, carbohydrates, amino acids, fatty acids, surfactants, and bulking agents, and are known to those skilled in the art. An acidic or a basic excipient is also suitable. The amount of excipient used is based on ratio to the biologically active labile agent, on a weight basis. For amino acids, fatty acids and carbohydrates, such as sucrose, trehalose, lactose, mannitol, dextran and heparin, the ratio of carbohydrate to biologically active labile agent, is typically between about 1:10 and about 20:1. For surfactants the ratio of surfactant to biologically active labile agent is typically between about 1:1000 and about 2:1. Bulking agents typically comprise inert materials. Suitable bulking agents are known to those skilled in the art.

The excipient can also be a metal cation component which is separately dispersed within the polymer matrix. This metal cation component acts to modulate the release of the biologically active labile agent and is not complexed with the biologically active agent. The metal cation component can optionally contain the same species of metal cation, as is contained in the metal cation stabilized biologically active labile agent, if present, and/or can contain one or more different species of metal cation. The metal cation component acts to modulate the release of the biologically active labile agent from the polymer matrix of the sustained release composition and can enhance the stability of the biologically active labile agent in the composition. A metal cation component used in modulating release typically comprises at least one type of multivalent metal cation. Examples of metal cation components suitable to modulate release include or contain, for example, Mg(OH)₂, MgCO₃ (such as 4MgCO₃.Mg(OH)₂.5H₂O), MgSO₄, Zn(OAc)₂, Mg(OAc)₂, ZnCO₃ (such as 3Zn(OH)₂.2ZnCO₃), ZnSO₄, ZnCl₂, MgCl₂, CaCO₃, Zn₃(C₆H₅O₇)₂ and Mg₃(C₆H₅O₇)₂. A suitable ratio of metal cation component to polymer is between about 1:99 to about 1:2 by weight. The optimum ratio depends upon the polymer and the metal cation component utilized. A polymer matrix containing a dispersed metal cation component to modulate the release of a biologically active labile agent from the polymer matrix is further described in U.S. Pat. No. 5,656,297 to Bernstein et al. and co-pending U.S. patent application Ser. No. 09/056,566 filed on Apr. 7, 1998, the teachings of both of which are incorporated herein by reference in their entirety.

The invention described herein also relates to pharmaceutical compositions suitable for use in the invention. In one embodiment, the pharmaceutical composition comprises a sustained release composition comprising a biocompatible polymer having an effective amount of a biologically active labile agent incorporated therein, and a corticosteroid. It is preferred that the labile agent is release for a period of at least bout two weeks. For example, release of the agent can be for a period of at least about three weeks, such as at least about four weeks. The corticosteroid is present in an amount sufficient to modify the release profile of the biologically active labile agent from the sustained release composition.

In one embodiment, the corticosteroid can be co-incorporated into the sustained release composition comprising the biocompatible polymer and the biologically active labile agent incorporated therein.

In another embodiment, the pharmaceutical composition comprises the sustained release composition comprising a first biocompatible polymer having incorporated therein an effective amount of a biologically active labile agent and a second biocompatible polymer having incorporated therein the corticosteroid. In a particular embodiment, the first and second polymers are the same type of polymer. In another embodiment, the first and second polymers are different.

In yet another embodiment, the corticosteroid can be present in the pharmaceutical composition in an unencapsulated state. For example, the corticosteroid can be commingled with the sustained release composition. In one embodiment, the corticosteroid can be solubilized in the vehicle used to deliver the pharmaceutical composition. Alternatively, the corticosteroid can be present as a solid suspended in an appropriate vehicle useful for delivering the pharmaceutical composition. Particular vehicles suitable for delivery are described in published PCT Application WO01/91720 to Ramstack et al. having a publication date of Dec. 6, 2001, the entire content of which is hereby incorporated by reference. Further, the corticosteroid can be present as a powder which is commingled with the sustained release composition.

A number of methods are known by which sustained release compositions (polymer/active labile agent matrices) can be formed. In many of these processes, the material to be encapsulated is dispersed in a solvent containing a wall forming material. At a single stage of the process, solvent is removed from the microparticles and thereafter the microparticle product is obtained.

Methods for forming a composition for the sustained release of biologically active labile agent are described in U.S. Pat. No. 5,019,400, issued to Gombotz et al., and issued U.S. Pat. No. 5,922,253 issued to Herbert et al. the teachings of which are incorporated herein by reference in their entirety.

In this method, a mixture comprising a biologically active labile agent, a biocompatible polymer and a polymer solvent is processed to create droplets, wherein at least a significant portion of the droplets contains polymer, polymer solvent and the biologically active labile agent. These droplets are then frozen by a suitable means. Examples of means for processing the mixture to form droplets include directing the dispersion through an ultrasonic nozzle, pressure nozzle, Rayleigh jet, or by other known means for creating droplets from a solution.

Means suitable for freezing droplets include directing the droplets into or near a liquified gas, such as liquid argon or liquid nitrogen to form frozen microdroplets which are then separated from the liquid gas. The frozen microdroplets are then exposed to a liquid or solid non-solvent, such as ethanol, hexane, ethanol mixed with hexane, heptane, ethanol mixed with heptane, pentane or oil.

The solvent in the frozen microdroplets is extracted as a solid and/or liquid into the non-solvent to form a polymer/active labile agent matrix comprising a biocompatible polymer and a biologically active labile agent. Mixing ethanol with other non-solvents, such as hexane, heptane or pentane, can increase the rate of solvent extraction, above that achieved by ethanol alone, from certain polymers, such as poly(lactide-co-glycolide) polymers.

A wide range of sizes of sustained release compositions can be made by varying the droplet size, for example, by changing the ultrasonic nozzle diameter. If the sustained release composition is in the form of microparticles, and very large microparticles are desired, the microparticles can be extruded, for example, through a syringe directly into the cold liquid. Increasing the viscosity of the polymer solution can also increase microparticle size. The size of the microparticles which can be produced by this process ranges, for example, from greater than about 1000 to about 1 micrometers in diameter.

Yet another method of forming a sustained release composition, from a suspension comprising a biocompatible polymer and a biologically active labile agent, includes film casting, such as in a mold, to form a film or a shape. For instance, after putting the suspension into a mold, the polymer solvent is then removed by means known in the art, or the temperature of the polymer suspension is reduced, until a film or shape, with a consistent dry weight, is obtained.

A further example of a conventional microencapsulation process and microparticles produced thereby is disclosed in U.S. Pat. No. 3,737,337, incorporated by reference herein in its entirety, wherein a solution of a wall or shell forming polymeric material in a solvent is prepared. The solvent is only partially miscible in water. A solid or core material is dissolved or dispersed in the polymer-containing mixture and, thereafter, the core material-containing mixture is dispersed in an aqueous liquid that is immiscible in the organic solvent in order to remove solvent from the microparticles.

Another example of a process in which solvent is removed from microparticles containing a substance is disclosed in U.S. Pat. No. 3,523,906, incorporated herein by reference in its entirety. In this process a material to be encapsulated is emulsified in a solution of a polymeric material in a solvent that is immiscible in water and then the emulsion is emulsified in an aqueous solution containing a hydrophilic colloid. Solvent removal from the microparticles is then accomplished by evaporation and the product is obtained.

In still another process as shown in U.S. Pat. No. 3,691,090, incorporated herein by reference in its entirety, organic solvent is evaporated from a dispersion of microparticles in an aqueous medium, preferably under reduced pressure.

Similarly, the disclosure of U.S. Pat. No. 3,891,570, incorporated herein by reference in its entirety, shows a method in which solvent from a dispersion of microparticles in a polyhydric alcohol medium is evaporated from the microparticles by the application of heat or by subjecting the microparticles to reduced pressure.

Another example of a solvent removal process is shown in U.S. Pat. No. 3,960,757, incorporated herein by reference in its entirety.

Tice et al., in U.S. Pat. No. 4,389,330, describe the preparation of microparticles containing an active agent by a method comprising: (a) dissolving or dispersing an active agent in a solvent and dissolving a wall forming material in that solvent; (b) dispersing the solvent containing the active agent and wall forming material in a continuous-phase processing medium; (c) evaporating a portion of the solvent from the dispersion of step (b), thereby forming microparticles containing the active agent in the suspension; and (d) extracting the remainder of the solvent from the microparticles.

Without being bound by a particular theory it is believed that the release of the biologically active labile agent can occur by two different mechanisms. First, the biologically active labile agent can be released by diffusion through aqueous filled channels generated in the polymer matrix, such as by the dissolution of the biologically active labile agent, or by voids created by the removal of the polymer solvent during the preparation of the sustained release composition. A second mechanism is the release of the biologically active labile agent, due to degradation of the polymer. The rate of degradation can be controlled by changing polymer properties that influence the rate of hydration of the polymer. These properties include, for instance, the ratio of different monomers, such as lactide and glycolide, comprising a polymer; the use of the L-isomer of a monomer instead of a racemic mixture; and the molecular weight of the polymer. These properties can affect hydrophilicity and crystallinity, which control the rate of hydration of the polymer.

By altering the properties of the polymer, the contributions of diffusion and/or polymer degradation to biologically active labile agent release can be controlled. For example, increasing the glycolide content of a poly(lactide-co-glycolide) polymer and decreasing the molecular weight of the polymer can enhance the hydrolysis of the polymer and thus, provides an increased biologically active labile agent release from polymer erosion.

The composition of this invention can be administered in vivo, for example, to a human, or to an animal, orally, or parenterally such as by injection, implantation (e.g., subcutaneously, intramuscularly, intraperitoneally, intracranially, and intradermally), administration to mucosal membranes (e.g., intranasally, intravaginally, intrapulmonary, buccally or by means of a suppository), or in situ delivery (e.g., by enema or aerosol spray) to provide the desired dosage of labile agent based on the known parameters for treatment with the particular agent of the various medical conditions.

While this invention has been particularly shown and described with references to preferred embodiments thereof, it will be understood by those skilled in the art that various changes in form and details may be made therein without departing from the scope of the invention encompassed by the appended claims.

### EXEMPLIFICATIONS

### Materials and Methods

Animals

Except as noted, male Sprague-Dawley Rats, weighing between 350 to 500 grams (Charles River Laboratories, Inc.) were used in the studies described herein following acclimation in standard animal housing for at least six days. For the majority of the studies described herein, the animals were acclimated for at least seven days.

Immunosuppression

Animals that were immunosuppressed prior to administration of microparticles were treated with cyclosporin (Sandimmune, Sandoz; CS) by administering 5 mg/kg of cyclosporin intraperitoneally daily for days 0-14 post administration (except Sunday) or days 0-6 and 8-13 following administration of the sustained release composition, and then 3 times per week thereafter.

Microparticle Administration

Administration of the biologically active labile agent-containing microparticles and corticosteroid is described in detail below for the specified study.

Preparation of EPO-Containing Microparticles

Microparticles containing recombinant human Erythropoietin (EPO) were made following the procedure described in U.S. Pat. No. 5,716,644 issued on Feb. 10, 1998 to Zale et al., the entire content of which is hereby incorporated by reference. Generally, the EPO-containing microparticles were prepared using a polymer purchased from Alkermes, Inc. of Cincinnati, Ohio having Cat No. 5050DL2.5A which is a poly(lactide-co-glycolide) 25 kD polymer having a lactide/glycolide ratio of 50:50 and an IV of 0.24 as measured in chloroform with 1% Mg(OH)₂ as an excipient in the polymer phase. Where indicated, hydrocortisone or triamcinolone acetonide (both purchased from Sigma) was added to the polymer phase resulting in the indicated nominal loads of each (0.25%, 2% and 14% hydrocortisone and triamcinolone at 2%). The EPO was obtained from either Johnson & Johnson, New Brunswick, N.J. or Biochemie and stabilized prior to encapsulation with ammonium sulfate as described in U.S. Pat. No. 5,716,644 using an EPO loading of about 1.6% w/w of the total weight of stabilized EPO in the microparticles.

Encapsulation Procedure for Exendin-Containing Microparticles

The exendin-containing microparticles described herein were prepared by a coacervation process which is described below.

Coacervation—W/O/O Process

The coacervation process, also referred to herein as a water-oil-oil (W/O/O) process, requires formation of a water-in-oil emulsion with aqueous drug and organic polymer solutions. An oil, typically a silicone oil, was then added to the water-in-oil emulsion to induce phase separation and to precipitate the polymer. The embryonic microparticles were then quenched in a solvent that removes the oil and polymer solvent.

Exendin-4 was encapsulated in PLG polymer using a water-oil-oil (W/O/O) emulsion system. The initial embryonic microparticles were formed in a W/O/O inner emulsion step after which they were subjected to coacervation and hardening steps. The microparticles were collected, dried and filled into vials. Further details of each step in the complete process is set forth below.

Inner Emulsion Formation

A water-in-oil emulsion was created using sonication. The water phase of the emulsion contained dissolved exendin-4 and various excipients in water. Typically, sucrose and ammonium sulfate were present as excipients but other excipients and combinations of excipients were investigated. The PLG phase contained polymer dissolved in methylene chloride.

Coacervation Formation

Coacervation was induced by adding silicone oil at a controlled rate to the inner emulsion with agitation, forming embryonic microparticles. The embryonic microparticles formed were relatively soft and required hardening.

Microparticle Hardening

The embryonic microparticles were added to a heptane/ethanol solvent mixture with gentle agitation. The solvent mixture hardened the embryonic microparticles. After hardening for about one hour at about 3° C., the solvent mixture was decanted and pure heptane was added at 3° C. and mixed for about one hour.

Microparticles Drying and Collection

After the hardening step, the microparticles were transferred and collected on a fine mesh pore-plate inside a drying chamber. A final heptane rinse of the hardening vessel was performed. The microparticles were dried with nitrogen gas over a four-day period with temperature ramping from about 3° C. to about 38° C.

In general, PLG was dissolved in methylene chloride. The inner water phase was prepared by dissolving the exendin-4, sucrose or sucrose and ammonium sulfate in water or an aqueous buffer. The aqueous solution was then injected into the polymer solution while probe sonicating. The resultant water/oil emulsion was then added to an emulsion reactor. Silicone oil (350 centiStokes) was slowly added to the reactor via peristaltic pump with stirring at about 1000 rpm. The mixture was then added to n-heptane. After stirring, for about two hours, the microparticles were isolated by filtration and vacuum dried overnight.

The IF-1 Formulation of Table 11 had a 1% exendin-4 load (50 mg/mL exendin-4), 1% sucrose (50 mg/mL sucrose) in 30 mM sodium acetate (pH 4-4.5) and 3A, 50:50 PLG [Poly(lactide-co-glycolide); 25 kD Mol. Wt.; IV=0.24 (dL/g)].

The SF-2 Formulation of Table 11 had a 3% exendin-4 (in water), 2% sucrose and 0.5% ammonium sulfate in 4A, 50:50 PLG [Pol(lactide-co-glycolide); Mol. Wt. 45-64 kD; IV=0.45-0.47 (dL/g)].

Cryogenic Process

The insulin-containing and hFSH-containing microparticles were prepared according to the process described in U.S. Pat. No. 5,922,253 issued to Herbert et al. and U.S. Pat. No. 5,019,400, issued to Gombotz et al., the entire teachings of both of which are hereby incorporated by reference.

The outline of the process steps is as follows:
Formation of a polymer solution by dissolving polymer in a suitable polymer solvent.
Addition of the protein lyophilizate to the polymer solution to form a polymer/protein mixture.
Optional homogenization of the polymer/protein mixture.
Atomization of the polymer/protein mixture by sonication or other means of droplet formation, and freezing of the droplets by contact with liquid nitrogen.
Extraction of the polymer solvent from the polymer/protein droplets into an extraction solvent (e.g., −80° C. ethanol), thereby forming particles comprising a polymer/protein matrix.
Isolation of the particles from the extraction solvent by filtration.
Removal of remaining solvent by evaporation.
Sizing of particles to provide injectable product.
   Insulin-Containing Microparticles

Insulin-containing microparticles were prepared using a polymer purchased from Alkermes, Inc. of Cincinnati, Ohio having Cat No. 5050DL2.5A which is a poly(lactide-co-glycolide) 25 kD polymer having a lactide/glycolide ratio of 50:50 and an IV of 0.24 as measured in chloroform. Insulin was recombinant human insulin purchased from Sigma, St. Louis, Mo. The nominal load of insulin was 10% (actual 5.8%).

hFSH-Containing Microparticles

The polymer used was a purchased from Alkermes, Inc. of Cincinnati, Ohio. The polymer is a poly(lactide-co-glycolide) with a 50:50 lactide; glycolide ratio with a Mol. Wt. of 10 kD and a carboxylic acid end group.

The protein lyophilizate was a stabilized FSH formulation having 10% FSH, 80% sucrose and 10% phosphate salts. The lyophilizate was prepared by adding solutions of the sucrose and sodium phosphate to the bulk drug. Each formulated solution was then spray-freeze dried to produce a lyophilized powder. The protein lyophilizate was loaded at 0.5% rhFSH based on the total dy weight of the sustained release composition.

Preparation of Triamcinolone-Containing Microparticles

Triamcinolone acetonide-containing microparticles (2% load) were prepared as follows: 42 mg of triamcinolone acetonide was dissolved in benzyl alcohol. The triamcinolone solution was then added to about 24.3 mL of a 6% PLG (purchased from Alkermes, Inc. of Cincinnati, Ohio having Cat No. 5050DL2.5A which is a poly(lactide-co-glycolide) 25 kD polymer having a lactide/glycolide ratio of 50:50 and an IV of 0.24 as measured in chloroform) solution in methylene chloride. The resulting homogenous solution was added to a stirring solution of 5% PVA. The stirring rate was raised until microscopic examination of the emulsion indicated that the diameter of the droplets was about 150-75 microns. The emulsion was then slowly added to stirring cold water. After about 45 minutes of stirring, the suspension was allowed to settle at 4° C. The microparticles were collected by filtration, washed with cold water, frozen and lyophilized to dryness.

Preparation of Placebo Microparticles

Placebo microparticles were prepared according to the process for preparation of the triamcinolone microparticles, but absent the triamcinolone.

Preparation of Hydrocortisone-Containing Microparticles

The hydrocortisone-containing microparticles were prepared according to the procedure detailed above for the triamcinolone microparticles with either a 2% or 20% load.

Preparation of Budesonide-Containing Microparticles

The budesonide-containing microparticles were prepared according to the procedure detailed above for the triamcinolone microparticles and had a 2.0 or 2.2% load.

Preparation of Dexamethasone-Containing Microparticles

The dexamethasone-containing microparticles were prepared according to the procedure detailed above for the triamcinolone microparticles and had 2% load.

### Example 1

### Pharmacological Effects of Hydrocortisone or Triamcinolone on Erythropoietin Release from Erythropoietin-Containing Microparticles Following Co-Administration

The pharmacokinetic (PK)/pharmacodynamic (PD) responses to erythropoietin (EPO) released from EPO-containing microparticles when co-administered with hydrocortisone acetate or triamcinolone diacetate in vivo to male Sprague-Dawley rats was determined. The total number of animals used was 16 with an average weight of 400-450 gms. The animals were acclimated for at least six days prior to testing.

Immunosuppression

The rats were immunosuppressed with cyclosporin (Sandimmune, Sandoz; CS) 5 mg/kg ip daily for days 0-14 (except Sunday) and 3 time per week thereafter. Animals received systemic hydrocortisone along with cyclosporin on days 0 and 1.

Microparticle Administration

Animals were anesthetized with 5% halothane. Each animal was shaved and the back swabbed with alcohol. EPO-containing microparticles, previously vialed with hydrocortisone acetate (Sigma Fine Chemicals, Cat. No. 86H1304) or triamcinolone diacetate (Sigma Fine Chemicals, Cat. No. 127F0812) according to Table 1 below, were resuspended using 0.75 mL vehicle (3% carboxymethylcellulose, 0.1% Tween 20, 0.9% NaCl, pH approximately 6). The microparticles were prepared as described above. The microparticles were injected into an interscapular site using a 21 gauge thinwall needle attached to a 1 mL syringe. Animals were dosed to receive a total of 10,000 U EPO either alone (Group A) or in combination with a total of 5 mg of hydrocortisone acetate (Group B), or 1 mg (Group C) or 5 mg (Group D) of triamcinolone diacetate. Animals were followed for 35 days post implantation.

TABLE 1
Administration of Hydrocortisone Acetate or Triamcinolone Diacetate and
EPO-containing Microparticles to Rats

| | | | | Concentration | Sample |
|---|---|---|---|---|---|
| | | | | of Secondary | Collection Time |
| Group | # Animals | EPO | Secondary Agent | Agent | points (days) |
| A. | 4 | 10,000 U | None | | pre-bleed, 1, 2, 4, |
| | | | | | 7, 10, 14, 17, 21, |
| | | | | | 24, 28, 31, and 35 |
| B. | 4 | 10,000 U | Hydrocortisone | 5 mg | same as above |
| | | | Acetate | | |
| C. | 4 | 10,000 U | Triamcinolone | 1 mg | same as above |
| | | | Diacetate | | |
| D. | 4 | 10,000 U | Triamcinolone | 5 mg | same as above |
| | | | Diacetate | | |

Evaluation Parameters

To evaluate EPO serum levels, serum samples (400 μL) were collected via tail vein on the following days relative to microparticle administration: pre-bleed, 1, 2, 4, 7, 10, 14, 17, 21, 24, 28, 31, and 35. After clotting, the samples were centrifuged and frozen at −70° C. Serum EPO levels were quantitated by ELISA (Genzyme) according to manufacturer's instructions (Cat. No. #80-3775-00).

Hematocrits were evaluated manually following centrifugation for 5 minutes at 8000 rpm (on four animals per group) using a capillary tube. Hematocrits were also determined at the following intervals relative to microparticle administration: pre-bleed, 1, 4, 7, 10, 14, 21, 28 and 35.

Results

EPO Serum Levels

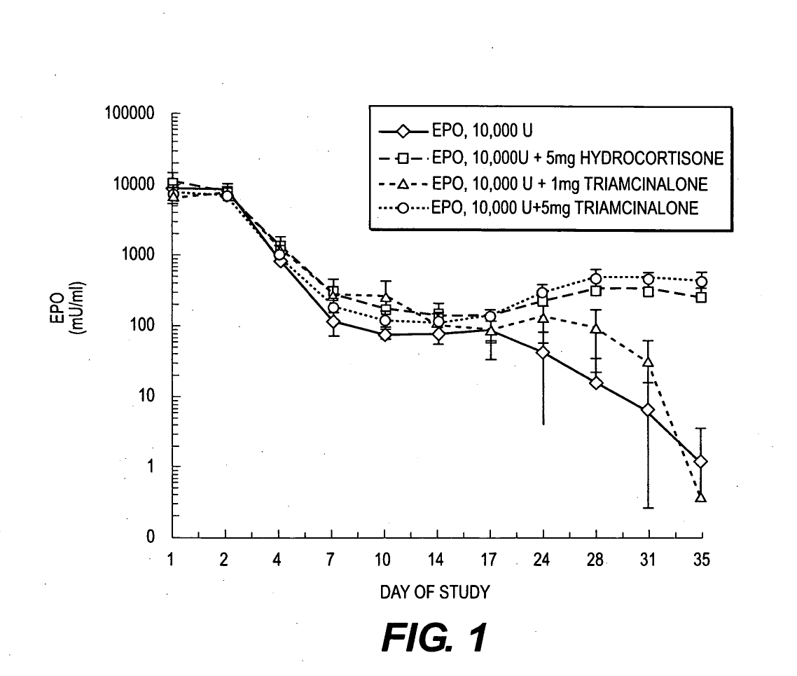

The results of the effect of the release of EPO from EPO-containing microparticles co-administered with hydrocortisone or triamcinolone to rats are shown in FIG. 1, which is a graph of serum EPO levels (mU/ml) in rats administered EPO-containing microparticles, EPO-containing microparticles admixed with hydrocortisone acetate (5 mg), or EPO-containing microparticles admixed with triamcinolone diacetate (1 mg or 5 mg) over time (days). As shown in FIG. 1, following an initial peak at about 10,000 mU/mL or above, serum EPO levels began to decrease steadily until day seventeen. By day twenty-four, a clear separation of treatment groups was observed. The EPO alone treated group (Group A) had dropped to 39.7 mU/mL±32.66 mU/mL. The groups that received a secondary agent in addition to the EPO-containing microparticles showed higher serum levels, at 210±32.66 mU/mL (Group B), 127.53±66.7 mU/mL (Group C) and 302.3±90.5 mU/mL (Group D). At day thirty-five, Groups A and C had dropped to below detection limits, but the two groups that had received either 5 mg hydrocortisone (Group B) or 5 mg triamcinolone (Group D) still had serum EPO levels of 241.5±43.9 mU/mL and 433.18±177.37 mU/mL, respectively.

These results indicate that co-administration of triamcinolone or hydrocortisone increased the duration of circulating EPO after release from EPO-containing microparticles.

Hematocrit Testing

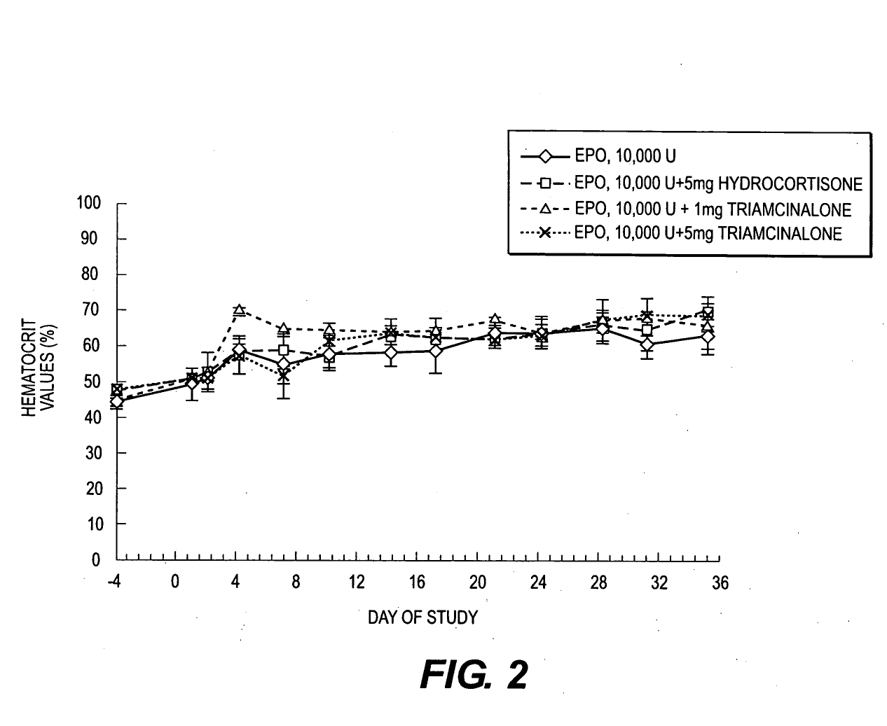

The results of hematocrit testing of rats administered EPO-containing microparticles, or co-administered EPO-containing microparticles with hydrocortisone or triamcinolone are shown in FIG. 2, which is a graph of hematocrit values (%) in rats administered EPO-containing microparticles, EPO-containing microparticles admixed with hydrocortisone acetate (5 mg), or EPO-containing microparticles admixed with triamcinolone diacetate (1 mg or 5 mg) over time (days). Hematocrit values increased steadily early in the study and reached a plateau by day 24 for all groups, when all animals had hematocrit values over 60%. There were no significant differences between groups for hematocrit values although values appeared to decrease in the animals receiving only EPO (Group A animals) at day 36. This was evidenced by the fact that hematocrit values in animals administered EPO-containing microparticles alone (Group A) and EPO-containing microparticles plus 1 mg triamcinolone (Group C) had decreased to mid-60%, while the groups receiving higher levels of hydrocortisone (Group B) or triamcinolone (Group D) had hematocrit values that were still at 70% or higher.

Histopathology

In rats treated with the high dose of triamcinolone diacetate (5 mg; Group D), the amount of residual polymer found at necropsy at day 35 was greater than in the animals administered hydrocortisone (5 mg; Group B) or a low dose (1 mg) of triamcinolone diacetate (Group C). Color of the skin overlying microsphere depots was pallid in most rats in Groups B and D. In addition, co-administration of hydrocortisone or triamcinolone diacetate with EPO-containing microparticles diminished the amount of peripheral fibrosis surrounding the microsphere depot in the subcutis and reduced the intensity of the granulomatous inflammatory reaction normally occurring within the microsphere mass.

These results indicate that triamcinolone and hydrocortisone decreased inflammation at the injection site.

### Example 2

### Administration of Microparticles Containing EPO and Hydrocortisone Coencapsulated and EPO-Containing Microparticles Co-Administered with Hydrocortisone

The pharmacodynamic and pharmacokinetic effects of the administration to immunodeficient nude rats (Tac:N:NIH-mufDF, Weight Range: 350-450 gm) of microparticles containing EPO and hydrocortisone coencapsulated at various levels (0, 0.25, 2 and 14%) and EPO-containing microparticles coadministered with hydrocortisone was determined.

Preparation of EPO-Containing Microparticles, and Microparticles Containing EPO and Hydrocortisone Co-Encapsulated

EPO-containing microparticles were prepared according procedure above. Microparticles containing hydrocortisone and EPO co-encapsulated at 0.25%, 2% and 14% [% refers to nominal hydrocortisone load (w/w)] were prepared as described above. Hydrocortisone coadministered was purchased from Sigma, St. Louis, Mo.

Administration of Microparticles

Microparticle were administered as described in Example 1 and as summarized in Table 2. Animals were dosed to receive a total of 10,000 Units of EPO-containing microparticles (Group 1), EPO co-encapsulated with 0.25% hydrocortisone (Group 2), 2% hydrocortisone (Group 3), 14% hydrocortisone (Group 4) or 5 mg of hydrocortisone coadministered. An untreated group (Group 6) was also included in this study. Sample collection time points were pre-bleed, 1, 5, 8, 12, 15, 19, 22, 26, 29, 34, 41, 48 and 55 days.

TABLE 2

| | | | | | | Sample |
|---|---|---|---|---|---|---|
| | | EPO | | | Microparticles | Collection Time |
| Group | # Animals | (units/dose) | | Treatments | (mg/dose) | points (days) |
| 1 | 4 | 10,000 | U | EPO Microparticles | 15 | pre-bleed, 1, 5, 8, |
| | | | | | | 12, 15, 19, 22, 26, |
| | | | | | | 29, 34, 41, 48 and |
| | | | | | | 55 |
| 2 | 4 | 10,000 | U | EPO microparticles | 15 | same as above |
| | | | | with 0.25% HC coencapsulated | | |
| 3 | 4 | 10,000 | U | EPO microparticles | 15 | same as above |
| | | | | with 2% HC coencapsulated | | |
| 4 | 3 | 10,000 | U | EPO microparticles | 15 | same as above |
| | | | | with 14% HC coencapsulated | | |
| 5 | 3 | 10,000 | U | EPO microparticles | 15 | same as above |
| | | | | and 5 mg HC | | |
| | | | | coadministered | | |
| 6 | 2 | 0 | | Untreated | 0 | same as above |

Evaluation Parameters

To evaluate EPO serum levels, serum samples (400 μL) were collected via tail vein on the days specified in Table 2. After clotting, the samples were centrifuged for about 5 minutes at about 13000 rpm and frozen at −70° C. Serum EPO levels were quantitated by ELISA (Genzyme), according to manufacturer's instruction (Cat. No. 80-3775-00).

Hematocrits were evaluated manually following centrifugation for 5 minutes at 8000 rpm (three animals per group) using a capillary tube. Hematocrits were also determined at the timepoints set forth in Table 2.

Results

EPO Serum Levels

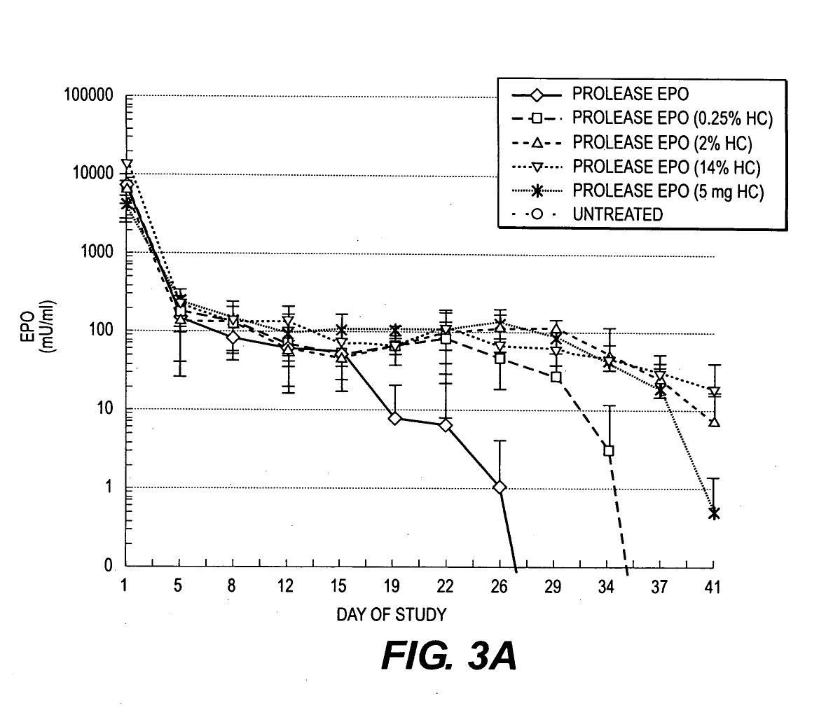

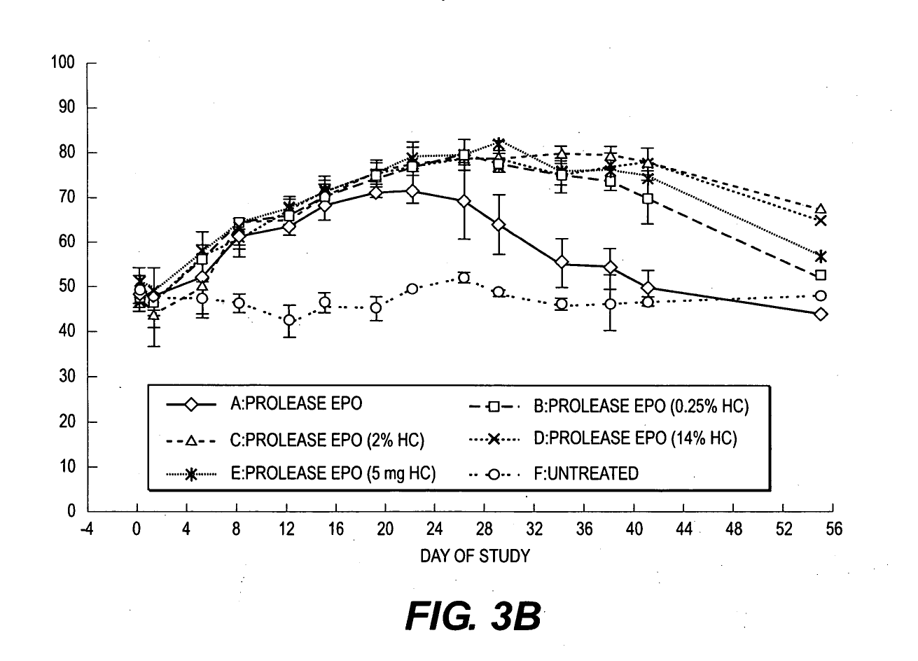

The results of the effect of the release of EPO from EPO-containing microparticles co-encapsulated or co-administered with hydrocortisone are shown in FIG. 3A, which is a graph of serum EPO levels (mU/ml) in rats administered microparticles containing EPO co-encapsulated with hydrocortisone at various levels and EPO-containing microparticles admixed with hydrocortisone acetate (5 mg) versus time in days. As shown in FIG. 3A, all treatments groups receiving hydrocortisone, either co-encapsulated or coadministered, exhibited an increase in the circulating EPO serum levels. More specifically, while serum EPO levels had decreased to non-detectable levels at day 26 in EPO only treated rats, levels did not reach non-detectable limits until day 34 in the low dose groups reveiving 0.25% of hydrocortisone. A dose-dependent increase in duration was seen as both groups with 2% and 14% hydrocortisone, respectively, had serum EPO levels of 10 mU/mL at day 41.

Hematocrit Testing

The results of the effect of the release of EPO from EPO-containing microparticles co-encapsulated or co-administered with hydrocortisone are shown in FIG. 3B, which is a graph of hematocrit values (%) in rats versus time (days) for the groups of Table 2. The graph shows that hematocrits remained low (45-50%) for untreated animals throughout the study; however, treated rats obtained hematocrit values reaching 60-70%. A return to baseline in hematocrits in rats receiving only EPO was observed on day 38, whereas all groups receiving EPO co-encapsulated with hydrocortisone did not return to baseline until at least day 56.

### Example 3

### EPO-Containing Microparticles Co-Administered with Hydrocortisone-Containing Microparticles or Admixed with Triamcinolone Acetonide

The pharmacodynamic and pharmacokinetic effects of the administration to rats of EPO-containing microparticles admixed with placebo microparticles, hydrocortisone-containing microparticles, or placebo microparticles admixed with triamcinolone acetonide, as well as the immunogenicity of such administration was determined.

Preparation of EPO-Containing Microparticles, Hydrocortisone-Containing Microparticles, and Placebo Microparticles admixed with Triamcinolone Acetonide

EPO-containing microparticles were prepared according to the procedure outlined above. Hydrocortisone-containing microparticles were prepared according to the procedure described above. Placebo microparticles were prepared according to the procedure outlined above.

Administration of Microparticles

Microparticle administration was as described in Example 1 and is summarized in Table 3. Animals were dosed to receive a total of 10,000 Units of EPO in combination with a total of 100 mg of placebo microparticles (Group A), 5 mg of triamcinolone acetonide and 100 mg of placebo microparticles admixed (Group B) and 100 mg of 20% w/w hydrocortisone-containing microparticles (Group C). Sample collection time points were pre-bleed, 1, 2, 6, 12, 19, and 26 days.

TABLE 3
Dosing of Rats Administered EPO-containing Microparticles and Secondary
Agents Contained in Microparticles or Admixed

| | | | | | Sample Collection Time |
|---|---|---|---|---|---|
| Group | # Animals | EPO | Secondary Agent | Treatment | Points (days) |
| A. | 4 | 10,000 U | Placebo | 100 mg | pre-bleed, 1, 2, 6, 12, 19, |
| | | | microparticles | | and 26 |
| B. | 4 | 10,000 U | Triamcinolone | 5 mg | same as above |
| | | (˜6.3 mg of | acetonide & | Triamcinolone | |
| | | microparticles) | placebo | acetonide & 100 mg | |
| | | | microparticles | placebo | |
| | | | | microparticles | |
| | | | | admixed | |
| C. | 4 | 10,000 U | 20% | 100 mg | same as above |
| | | | Hydrocortisone | | |
| | | | microparticles | | |

Serum Evaluation

To evaluate EPO serum levels, 0.4 mL samples were collected via tail vein on the days specified in Table 3 (four animals per group). After clotting, the samples were centrifuged and frozen (−70° C.). Serum EPO levels were quantitated by ELISA (R&D Systems), according to the manufacturer's instructions (Cat. No. DEP00, and the data were normalized for dose and body weight. Starting on day 12, serum samples were also assessed for EPO antibody levels weekly, using an ELISA This assay detects all antibody subclasses inasmuch as the detecting antibody is reactive with both immunoglobulin heavy (γ) and light chains. Hematocrit analyses were carried out as described in Example 1, and were tested at the following intervals relative to time of microsparticle administration: pre-bleed, 1, 2, 6, 12, 19, and 26 days.

Results

Serum EPO Levels

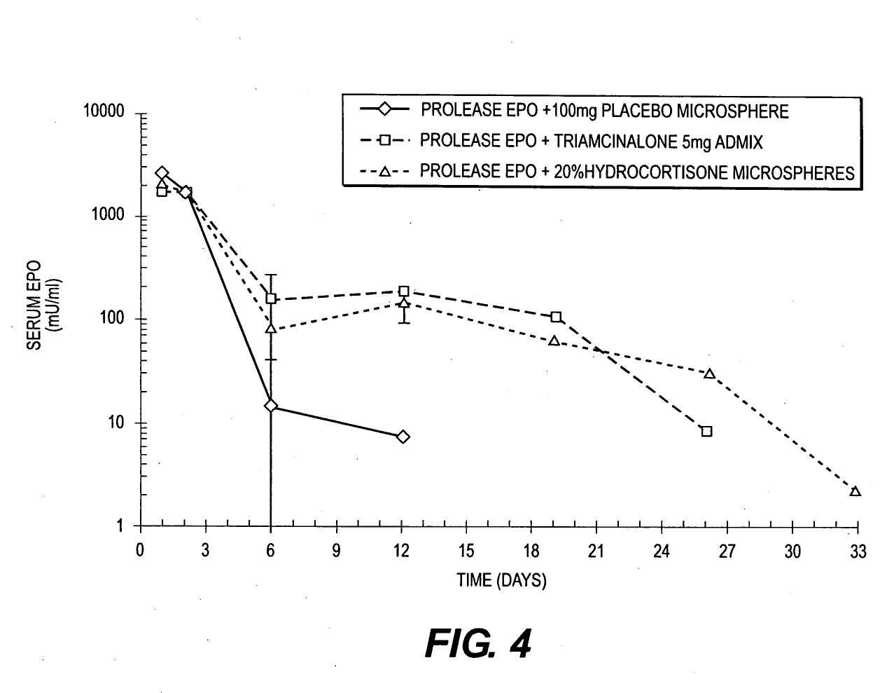

The release of EPO from EPO-containing microparticles admixed with placebo microparticles, triamcinolone acetonide admixed with placebo microparticles, hydrocortisone-containing microparticles is shown in FIG. 4, which is a graph of serum EPO levels (mU/mL) in rats administered each of the above formulations over time (days). As shown in FIG. 4, serum EPO levels diminished rapidly in the control group (animals administered EPO-containing microparticles and placebo microparticles; Group A), with no EPO detected after day 12.

The average serum EPO levels (steady state) between day 6 and day 19 were 148.6±102.9 mU/mL in animals administered EPO-containing microparticles plus triamcinolone admixed with placebo microparticles (Group B) compared to 7.23±7.12 mU/mL (p<0.05) the control animals of Group A. Following hydrocortisone microsphere treatment, the steady state values were 96.12±29.7 mU/mL (p<0.01).

Hematocrit Testing

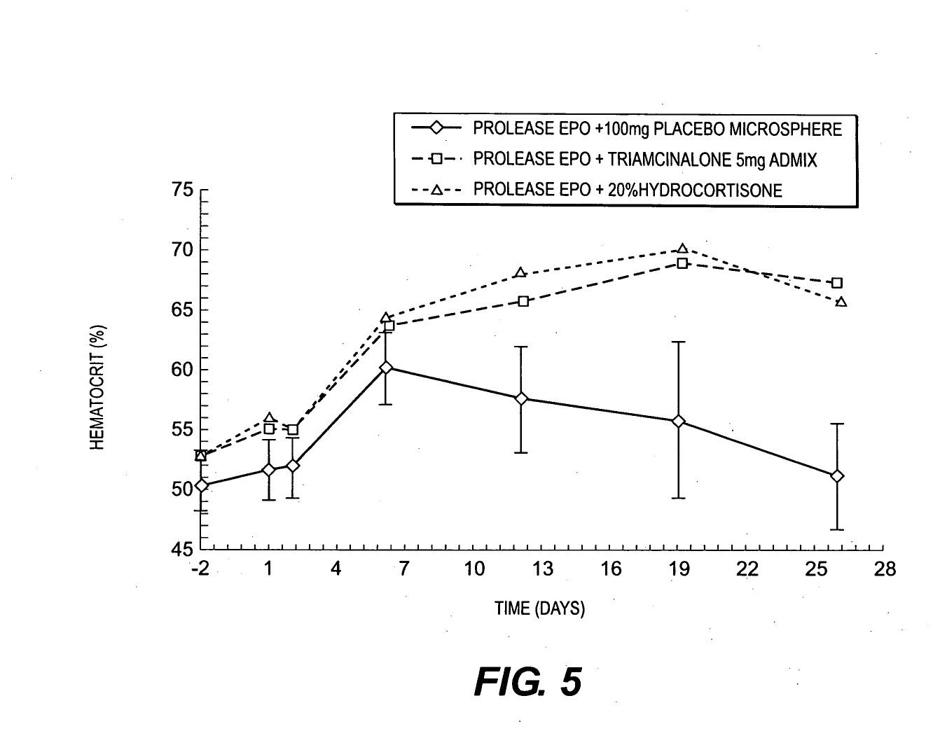

The results of administration of EPO-containing microparticles plus placebo microparticles, triamcinolone acetonide admixed with placebo microparticles and hydrocortisone-containing microparticles on hematocrit values are shown in FIG. 5, which is a graph of hematocrit values (%) in rats administered each of the above formulations over time (days). FIG. 5 represents the group average hematocrits for the entire study. The hematocrits for the control group receiving EPO-containing microparticles plus placebo microparticles (Group A) increased normally from day 0 through day 6. However, after day 6, there was a steady decline in hematocrit values, from 60.6%±3.11% on day 6 to 47.0%±3.56% on day 33. Animals in the groups administered EPO-containing microparticles plus corticosteroid reached hematocrit levels that were significantly higher than the control group by day 12. EPO microparticles co-administered with triamcinolone acetonide admixed with placebo microparticles induced hematocrit values to a maximum of 69.3%±3.3% which was significantly higher (p<0.05) than controls (56.00% 6.68) on day 19. EPO-containing microparticles loaded with 20% hydrocortisone (Group C) also helped to maintain higher hematocrits at 70.5%±1.91% (p<0.05). As such, the administration of hydrocortisone microparticles and admixed triamcinolone acetonide with placebo microparticles had comparable pharmacodynamic effects.

Immunogenicity of EPO-Containing Microparticles and Various Secondary Agents

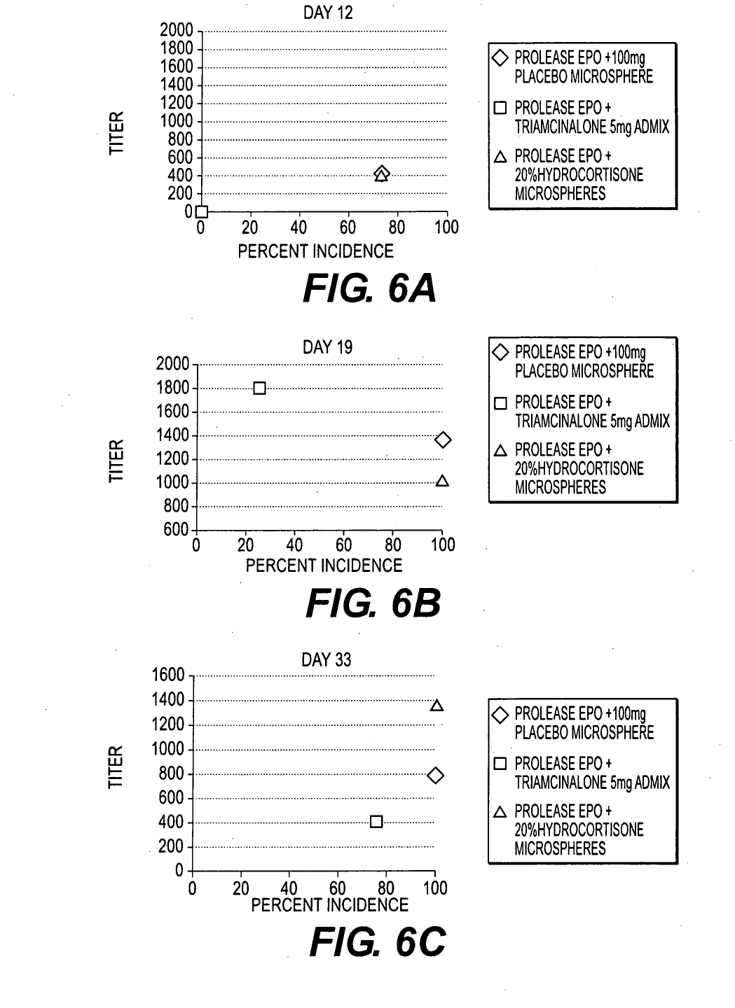

To assess the immune response evoked by EPO released from EPO-containing microparticles and the impact of the secondary agents on antibody production, sera were tested by ELISA for the presence and titer of anti-EPO antibody. The results of this assessment are shown in FIGS. 6A, 6B, and 6C (assayed at days 12, 19, and 33, respectively). The percent incidence versus geometric mean titer are presented in FIGS. 6A, 6B, and 6C, which are graphs of the incidence of antibodies to EPO (titer) detected in the serum of rats administered a total of 10,000 Units of EPO in combination with a total of 100 mg of placebo microparticles at day 12 (FIG. 6A) day 19 (FIG. 6B), and day 33 (FIG. 6C) after administration.

As shown in FIG. 6A, on day 12, control and hydrocortisone groups had between 75 and 100% of animals with some level of antibody (n=4/group) except the group that was treated with EPO-containing microparticles plus triamcinolone acetonide admixed with placebo microparticles (Group B). Additionally, by day 19 (FIG. 6B), only one animal in Group B had anti-EPO antibodies, with a titer of 1800. All the other groups had 100% of the animals with some level of anti-EPO antibody detected. By day 3 (FIG. 6C), the incidence in the triamcinolone treated Group B animals increased to 5% compared to an incidence of 100% in the other groups.

EPO antibody titer comparisons between groups showed that, with the exception of the triamcinolone acetonide treated Group B animals, titers were similar at day 12 (FIG. 6A; range: 288-600). By day 19, titers had increased in control and hydrocortisone groups, but not in the triamcinolone acetonide treated group (FIG. 6B). The titer in triamcinolone acetonide treated animals at day 33, was about 50% of the titer in the control group.

The results of these studies show that triamcinolone acetonide decreased antibody responses when co-administered with microparticles containing a protein to be delivered to a subject.

### Example 4

### EPO-Containing Microparticles Co-Administered with Dexamethasone-Containing, Budesonide-Containing and Triamcinolone Acetonide-Containing Microparticles

The pharmacodynamic and pharmacokinetic effects of the administration to rats of EPO-containing microparticles admixed with placebo microparticles, triamcinolone acetonide-containing microparticles, dexamethasone-containing microparticles and budesonide-containing microparticles was determined.

Preparation of Microparticles

EPO-containing microparticles were prepared according to the procedure described above. Dexamethasone-containing microparticles, budesonide-containing microparticles and triamcinolone acetonide-containing microparticles were prepared as described above. Placebo microparticles were prepared according to the procedure outlined above.

Immunosuppression

The rats were immunosuppressed with administration of cyclosporin (Sandimmune, Sandoz; CS), 5 mg/kg only ip daily, for 14 days (except Sundays) and three time/wk thereafter.

Administration of Microparticles

Microparticle administration was as described in Example 1 and is summarized in Table 4. Animals were dosed to receive a total of 10,000 Units of EPO in combination with the encapsulated corticosteroid as set forth in Table 4. Sample collection time points were pre-bleed, 1, 2, 5, 8, 12, 15, 19, 22, 26, 29, 33, and 36 days.

TABLE 4

| | | | | | Sample Collection |
|---|---|---|---|---|---|
| Group | # Animals | EPO | Secondary Agent | Treatment | Time Points (days) |
| A. | 4 | 10,000 U | Placebo | 10 mg | pre-bleed, 1, 2, 5, 8, |
| | | | microparticles | | 12, 15, 19, 22, 26, |
| | | | | | 29, 33 and 36 |
| B. | 4 | 10,000 U | 2% Triamcinolone | 10 mg | same as above |
| | | | acetonide | | |
| | | | microparticles | | |
| C. | 4 | 10,000 U | 2% Dexamethasone | 2.5 mg | same as above |
| | | | microparticles | | |
| D. | 4 | 10,000 U | 2% Budesonide | 10 mg | same as above |
| | | | microparticles | | |

Serum Evaluation

To evaluate EPO serum levels, 0.4 mL samples were collected via tail vein on the days specified in Table 4 (four animals per group). After clotting, the samples were centrifuged and frozen (−80° C.). Serum EPO levels were quantitated by ELISA (R&D Systems), according to the manufacturer's instructions (Cat. No. DEP00) and the data were normalized for dose and body weight.

Hematocrit analyses were carried out as described in Example 1, and were tested at the timepoints set forth in Table 4.

Results

Serum EPO Levels

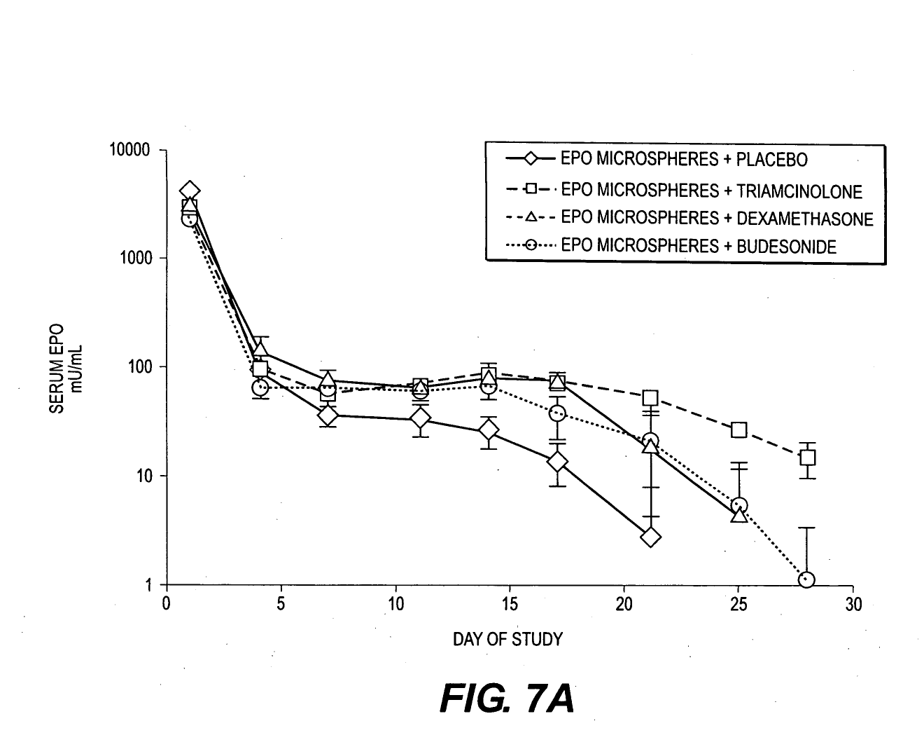

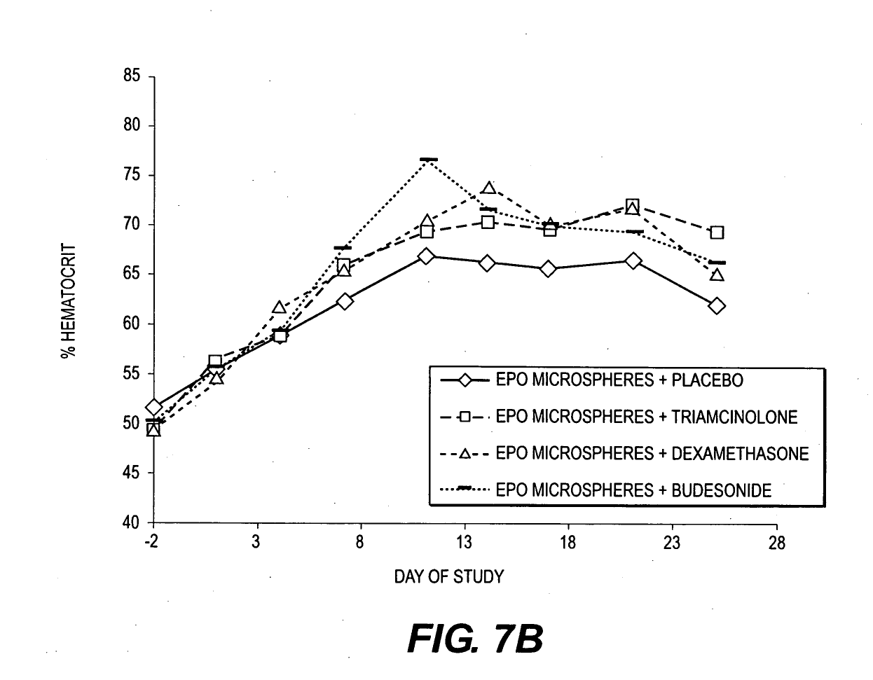

The release of EPO from EPO-containing microparticles admixed with placebo microparticles, dexamethasone-containing microparticles, budesonide containing microparticles and triamcinolone acetonide-containing microparticles is shown in FIG. 7A, which is a graph of serum EPO levels (mU/mL) in rats administered each of the above formulations over time (days). As shown in FIG. 7A, significant improvements in bioavailability as a result of coadministration of triamcinolone acetonide-, dexamethasone- and budesonide-containing microparticles with EPO-containing microparticles are realized with a notable extension of the duration of release. For example, the group treated with triamcinolone acetonide microparticles co-administered with EPO microparticles has the largest difference from control (placebo) in terms of duration of release and steady state values. The study was terminated at day 28, and at that time, there were still detectable serum levels of EPO in triamcinolone treated animals >12.5 mIU/mL. Steady state (day 7 to 25) values were significantly higher in this group compared to controls at 60.36 mIU/mL±7.7 mIU/mL versus 19.45±5.28 mIU/mL in controls (p<0.001). Both dexamethasone and budesonide also had significantly higher steady state values (day 7-25) over controls, at 55.2±10.7 mIU/mL and 43.7±9.8 mIU/mL (p<0.01).

Hematocrit Testing

Results of administration of EPO-containing microparticles admixed with placebo microparticles, dexamethasone-containing microparticles, budesonide containing microparticles and triamcinolone acetonide-containing microparticles on hematocrit values are shown in FIG. 7B, which is a graph of hematocrit values (%) in rats administered each of the above formulations over time (days). All of the groups were significantly higher than controls at the time points of the maximal hematocrit. For example, by day 11, hematocrit in placebos had reached its maximum at 67±2.2%. However, triamcinolone acetonide induced a maximal hematocrits response on day 21 at 72.5±4.4. Dexamethasone was seen to also increase hematocrit with the group average being highest on day 14 at 74.3±2.6%. Budesonide was also seen to increase hematocrit, and was 76.8%±2.5% on day 11.

### Example 5

### EPO-Containing Microparticles Co-Administered with Budesonide-Containing and Triamcinolone Acetonide-Containing Microparticles at Various Doses and Co-Encapsulated

The pharmacodynamic and pharmacokinetic effects of the administration to rats of EPO-containing microparticles admixed with placebo microparticles, triamcinolone acetonide-containing microparticles, and budesonide-containing microparticles as well as microparticles having EPO and triamcinolone co-encapsulated was determined.

Preparation of Microparticles

EPO-containing microparticles were prepared according to the procedure described above. Budesonide-containing microparticles and triamcinolone-containing microparticles were prepared as described above. Placebo microparticles were prepared according to the procedure described above. Microparticles having EPO and triamcinolone co-encapsulated were prepared as described above.

Immunosuppression

The rats were immunosuppressed with administration of cyclosporin (Sandimmune, Sandoz; CS), 5 mg/kg only ip daily, for 14 days (except Sundays) and three time/wk thereafter.

Administration of Microparticles

Microparticle administration was as described in Example 1 and is summarized in Table 5. Animals were dosed to receive a total of 10,000 Units of EPO coencapsulated with triamcinolone acetonide or in combination with separately encapsulated corticosteroid as set forth in Table 5. Sample collection time points were pre-bleed, 1, 2, 5, 8, 12, 15, 19, 22, 26, 29, 33 and 36 days.

TABLE 5

| | | | | | Sample Collection |
|---|---|---|---|---|---|
| Group | # Animals | EPO | Secondary Agent | Treatment | Time Points (days) |
| A. | 4 | 10,000 U | Placebo | 50 mg | pre-bleed, 1, 2, 5, 8, |
| | | | microparticles | | 12, 15, 19, 22, 26, |
| | | | | | 29, 33 and 36 |
| B. | 4 | 10,000 U | 2% Triamcinolone | 5 mg | same as above |
| | | | acetonide | | |
| | | | microparticles | | |
| C. | 4 | 10,000 U | 2% Triamcinolone | 10 mg | same as above |
| | | | acetonide | | |
| | | | microparticles | | |
| D. | 4 | 10,000 U | 2% Triamcinolone | 20 mg | same as above |
| | | | acetonide | | |
| | | | microparticles | | |
| E. | 4 | 10,000 U | 2% Triamcinolone | | same as above |
| | | | coencapsulated | | |
| F. | 4 | 10,000 U | 2.2% Budesonide | 25 mg | same as above |
| | | | microparticles | | |
| G. | 4 | 10,000 U | 2.2% Budesonide | 50 mg | same as above |
| | | | microparticles | | |

Serum Evaluation

To evaluate EPO serum levels, 0.4 mL samples were collected via tail vein on the days 1 through 7, and 0.5 mL on remaining days specified in Table 5 (four animals per group). After clotting, the samples were centrifuged and frozen (−80° C.). Serum EPO levels were quantitated by ELISA (R&D Systems), according to the manufacturer's instructions (Cat. No. DEP00), and the data were normalized for dose and body weight.

Hematocrit analyses were carried out as described in Example 1, and were tested at the timepoints set forth in Table 5.

Results

Serum EPO Levels

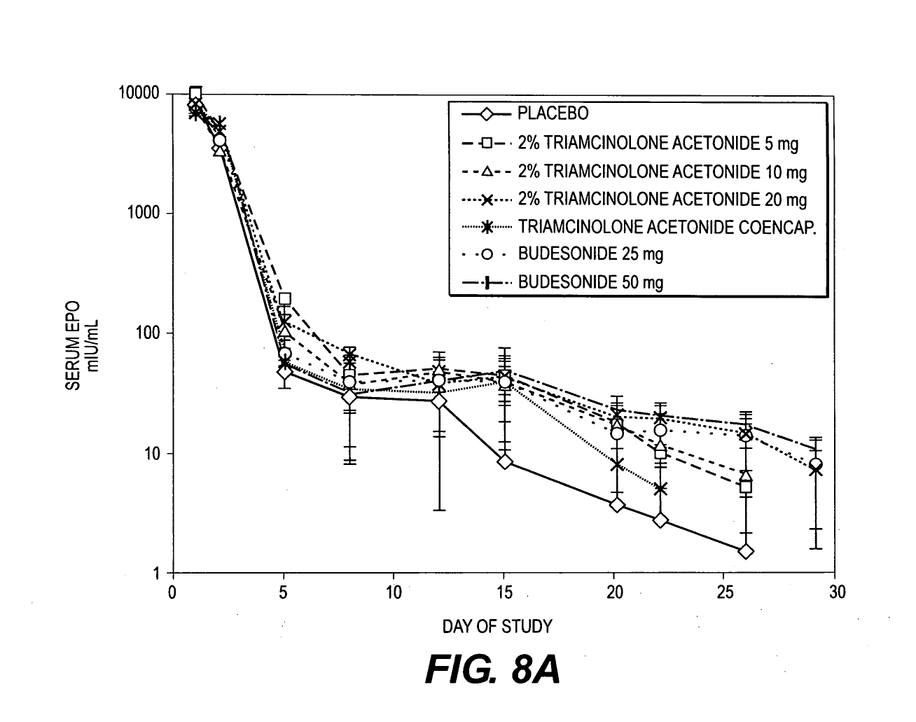

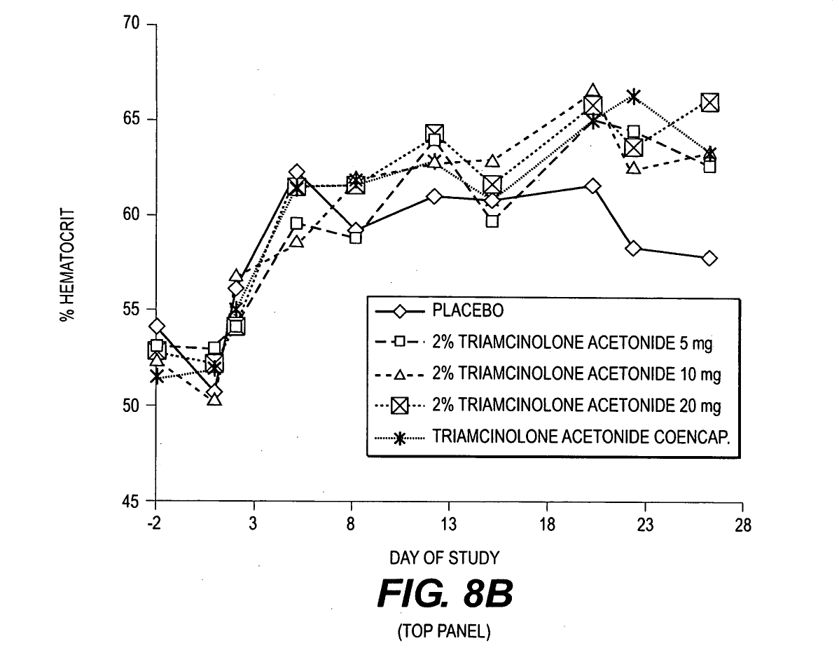

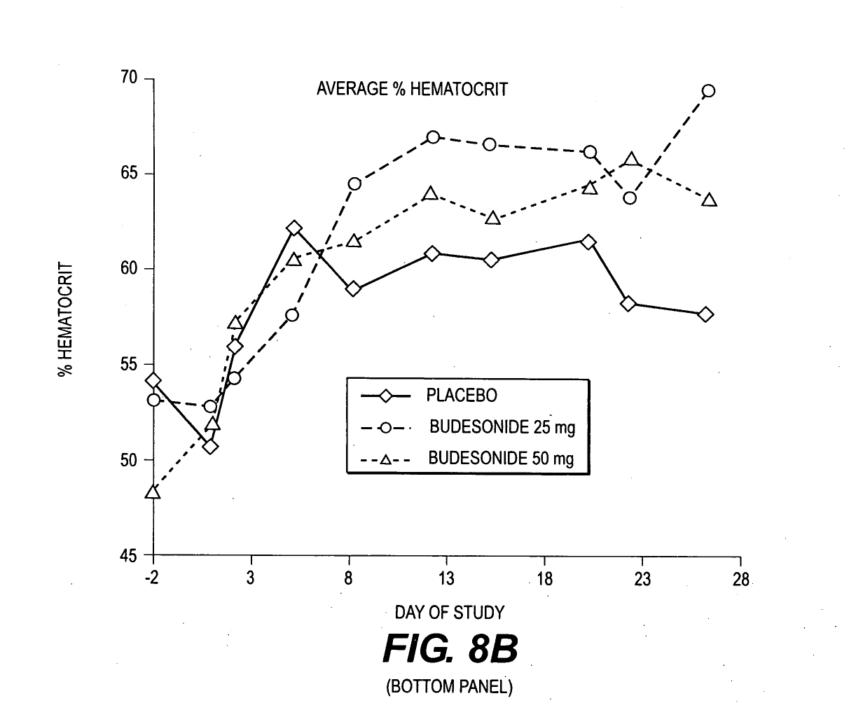

The release of EPO from EPO-containing microparticles admixed with placebo microparticles, triamcinolone acetonide-containing microparticles, and budesonide-containing microparticles as well as microparticles having EPO and triamcinolone acetonide co-encapsulated is shown in FIG. 8A, which is a graph of serum EPO levels (mU/mL) in rats administered each of the above formulations over time (days). As shown in FIG. 8A, both budesonide treated and triamcinolone treated animals exhibited an extension of the duration of release of EPO. For example, both the 25 mg and 50 mg budesonide groups and the 20 mg triamcinolone group had detectable levels of EPO until the termination of the study on day 29. At that time, the detectable serum level of EPO in triamcinolone treated animals was >14.0 mIU/mL, and the budesonide groups (25 mg, and 50 mg) had levels of 13.3 mIU/mL and 13.4 mIU/mL, respectively. All treatment groups showed significant increases in steady state serum levels (day 5 though day 22) and post burst (day 5 through day 33) AUCs (Area Under the Curve) were significantly enhanced.

Hematocrit Testing

Results of administration of EPO-containing microparticles admixed with placebo microparticles, triamcinolone-containing microparticles, and budesonide-containing microparticles as well as microparticles having EPO and triamcinolone co-encapsulated is shown in FIG. 8B, which is a graph of hematocrit values (%) in rats administered each of the above formulations over time (days). FIG. 8B shows that both triamcinolone and budesonide groups elevated packed blood cell volume in a comparable way.

### Example 6

### Effects of Local Delivery of Secondary Agent-Containing Microparticles on the Release of Follicle Stimulating Hormone from Follicle Stimulating Hormone-Containing Microparticles

The pharmacokinetic responses to human follicle stimulating hormone (hFSH) released from hFSH-containing microparticles when co-administered with hydrocortisone-containing microparticles or triamcinolone acetonide-containing microparticles in vivo to male Sprague-Dawley rats was determined.

Preparation of hFSH-containing microparticles, Hydrocortisone-Containing Microparticles, and Triamcinolone Acetonide-Containing Microparticles

Human FSH-containing microparticles were prepared according to the procedure described above. Hydrocortisone-containing microparticles were prepared according to the procedure described above. Triamcinolone-containing microparticles were prepared as described above. Placebo microparticles were prepared according to the procedure described above.

Administration of Microparticles

Microparticle administration and sample collection were conducted as described in Example 1. Treatment groups are summarized in Table 6. Animals were dosed to receive a total of 15 mg of hFSH-containing microparticles in combination with a total of 75 mg of placebo microparticles (Group A), 10 mg of 2% w/w triamcinolone microparticles (Group B), or 15 mg of 2% w/w hydrocortisone-containing microparticles (Group C). The rats in this study were immunosuppressed with cyclosporin (Sandimmune, Sandoz; CS), 5 mg/kg only ip daily (except Sundays), for 14 days and 3 times per week thereafter. Sample collection time points were pre-bleed, 6 hrs, 12 hrs, and days 1, 2, 4, 7, 10, 14, 17, 21, 24, 28, 31, 35 and 38.

TABLE 6
Administration of hFSH-containing Microparticles Co-administered with
Microparticles Containing a Secondary Agent

| | # | hFSH | Secondary | | Sample Collection |
|---|---|---|---|---|---|
| Group | Animals | Microparticles | Agent | Treatment | Time points (days) |
| A. | 4 | 15 mg | Placebo | 75 mg | pre-bleed, 6 hrs, 12 hrs, |
| | | | microparticles | | day 1, 2, 4, 7, 10, 14, |
| | | | | | 17, 21, 24, 28, 31, 35 |
| | | | | | and 38 |
| B. | 4 | 15 mg | 2% Triamcinolone | 10 mg | same as above |
| | | | acetonide | | |
| | | | microparticles | | |
| C. | 4 | 15 mg | 2% hydrocortisone | 15 mg | same as above |
| | | | microparticles | | |

Evaluation Parameters
Serum hFSH Levels

To measure serum hFSH levels, 0.4 mL of serum were collected via tail vein on the days specified in Table 6 (four animals per group). After clotting, the samples were centrifuged and frozen (−70° C.). Serum hFSH levels were quantitated by ELISA according to manufacturer's instructions (American Research Products; Cat. No. P-2035).

Results

Serum hFSH Levels

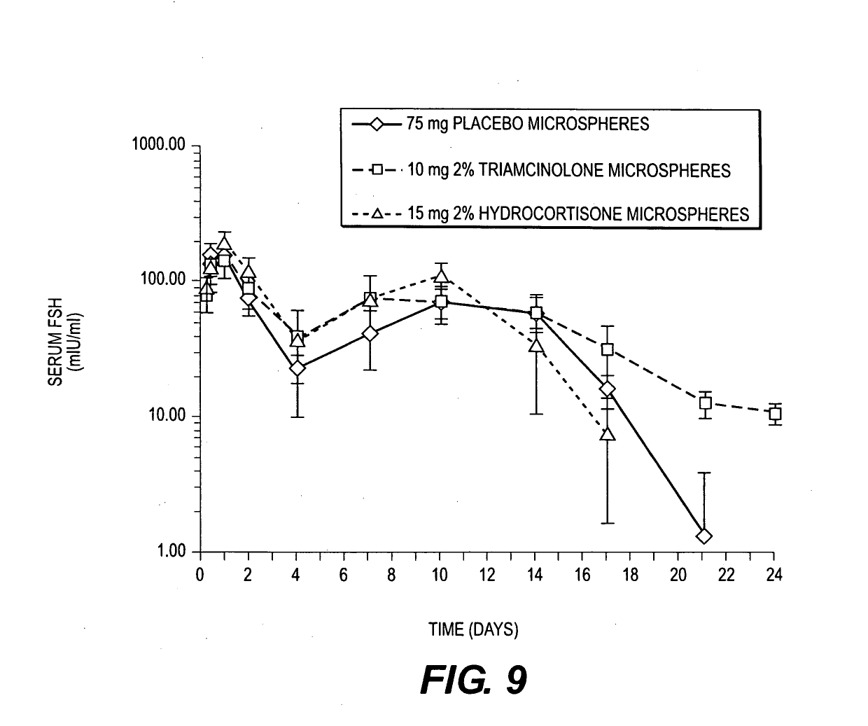

Serum samples were collected as indicated in Table 6 following administration of hFSH-containing microparticles co-administered with either placebo microparticles hydrocortisone-containing microparticles or triamcinolone acetonide-containing microparticles, and tested by ELISA for serum hFSH levels according to manufacturer's instructions (American Research Products; Cat. No. P-2035). FIG. 9 shows the pharmacokinetic profile for each group over the course of the study in the form of a graph of serum hFSH levels (mIU/mL) in rats administered hFSH-containing microparticles in combination with a total of 75 mg of placebo microparticles, 10 mg of 2% w/w triamcinolone acetonide microparticles, or 15 mg of 2% w/w hydrocortisone-containing microparticles over time (days). As shown in FIG. 9, there were no significant differences during the burst phase in serum levels of hFSH, with Cₘₐₓ values ranging from 140.8±35.2 mIU/mL to 200.3±35.3 mIU/mL. The hFSH release profile showed a biphasic curve in all the groups, with serum levels decreasing by day 4, and increasing again to peak at day 10. Day 10 serum levels of hFSH in rats treated with hydrocortisone-containing microparticles (Group C) were the highest at 114.1±18.9 mIU/mL, although this level was not significantly different from levels in rats receiving placebo microparticles (Group A) (69.0±20.1 mIU/mL). By day 21, serum hFSH levels in the hydrocortisone treated animals, Group C, had dropped below detectable limits. The control Group A animals had serum levels of 1.3±2.6 mIU/mL by day 21 and was also below detectable levels by day 24.

However, serum levels in all rats treated with hFSH-containing microparticles co-administered with triamcinolone acetonide-containing microparticles (Group B) had serum levels, about 10 mIU/mL at day 24, whereupon the animals were euthanized for injection site analysis. The serum levels were significantly higher at day 21-day 24 as compared to control animals.

The results suggests that triamcinolone can be more effective than hydrocortisone at comparable doses at extending the duration of release of therapeutically effective levels of FSH.

### Example 7

### Effects of Local Delivery of Secondary Agent-Containing Microparticles on the Release of Follicle Stimulating Hormone from Follicle Stimulating Hormone-Containing Microparticles

The pharmacokinetic response to human follicle stimulating hormone (hFSH) released from hFSH-containing microparticles when co-administered with triamcinolone acetonide-containing microparticles in vivo to male Sprague-Dawley rats was determined.

Preparation OF hFSH-Containing Microparticles, Hydrocortisone-Containing Microparticles, and Triamcinolone-Containing Microparticles

Human FSH-containing microparticles were prepared according to the procedure described above. Triamcinolone acetonide-containing microparticles were prepared as described above. Placebo microparticles were prepared according to the procedure described above.

Administration of Microparticles

Microparticle administration and sample collection were conducted as described in Example 1. Treatment groups are summarized in Table 7. Animals were dosed to receive a total of 15 mg of hFSH-containing microparticles in combination with a total of 100 mg of placebo microparticles (Group A) and 10 mg of 2% w/w triamcinolone microparticles with 90 mg of placebo microparticles (Group B). The rats in this study were immunosuppressed with cyclosporin (Sandimmune, Sandoz; CS), 5 mg/kg only ip daily (except Sundays), for 14 days and 3 times per week thereafter. Sample collection time points were pre-bleed, 6 hrs, 12 hrs, and days 1, 2, 4, 7, 10, 14, 17, 21, 23, 27 and 30.

TABLE 7
Administration of hFSH-containing Microparticles Co-administered with
Microparticles Containing a Secondary Agent

| | | hFSH | | | Sample Collection |
|---|---|---|---|---|---|
| Group | # Animals | Microparticles | Secondary Agent | Treatment | Time points (days) |
| A. | 4 | 15 mg | Placebo | 100 mg | pre-bleed, 6 hrs, 12 hrs, |
| | | | microparticles | | day 1, 2, 4, 7, 10, 14, |
| | | | | | 17, 21, 23, 27 and 30 |
| B. | 4 | 15 mg | 2% Triamcinolone | 10 mg | same as above |
| | | | acetonide | | |
| | | | microparticles | | |
| | | | and | | |
| | | | 90 mg of placebo | | |
| | | | microparticles | | |

Evaluation Parameters
Serum hFSH Levels

To measure serum hFSH levels, 0.4 mL of serum were collected via tail vein on, the days specified in Table 7 (four animals per group). After clotting, the samples were centrifuged and frozen (−70° C.). Serum hFSH levels were quantitated by ELISA according to manufacturer's instructions (American Research Products; Cat. No. P-2035).

Results

Serum hFSH Levels

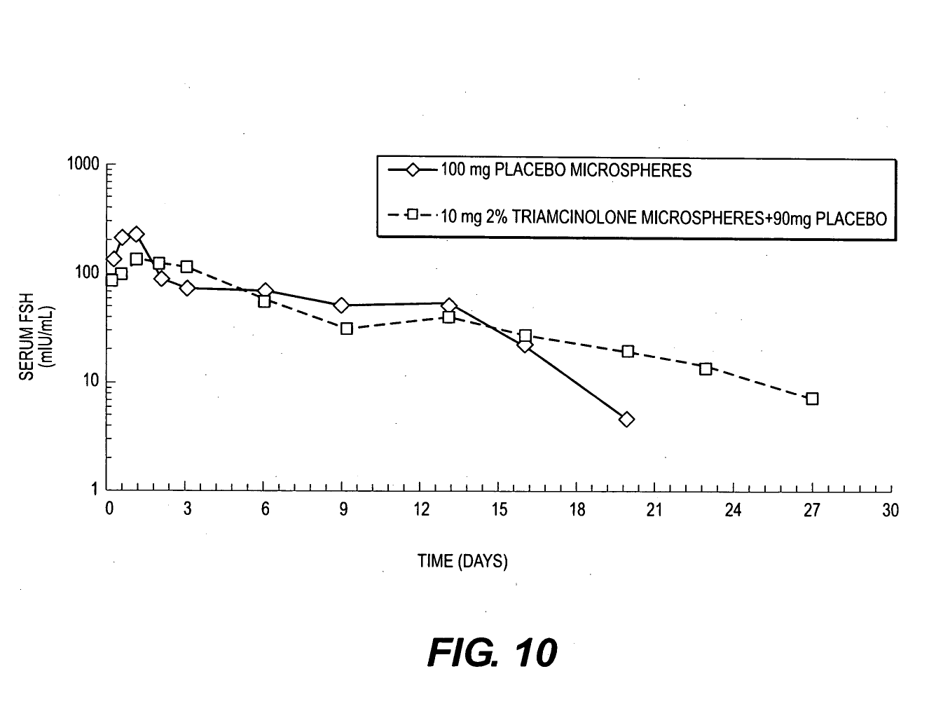

Serum samples were collected as indicated in Table 7 following administration of hFSH-containing microparticles co-administered with either placebo microparticles, or triamcinolone acetonide-containing microparticles plus placebo, and tested by ELISA for serum hFSH levels according to manufacturer's instructions (American Research Products; Cat. No. P-2035). FIG. 10 shows the pharmacokinetic profile for each group over the course of the study in the form of a graph of serum hFSH levels (mIU/mL) in rats administered hFSH-containing microparticles in combination with a total of 100 mg of placebo microparticles or 10 mg of 2% w/w triamcinolone acetonide microparticles and 90 mg of placebo microparticles over time (days). As shown in FIG. 10, the triamcinolone acetonide treated animals exhibited a significant decrease in serum FSH levels as compared to Group A (FSH microparticles alone) from 6 hours up to the day 3 timepoint. For example, at the 10 hour time point the serum FSH level of Group A was 218.3±56.6 mIU/mL while it was only 102.2±17.6 mIU/mL in the triamcinolone acetonide treated group. In addition, the overall release profile of the triamcinolone treated group exhibited a significant increase in serum FSH levels as compared to the control group on day 20.

### Example 8

### Effects of Local Delivery of Secondary Agents on the Release of Insulin from Insulin-Containing Microparticles

The effects of hydrocortisone and triamcinolone acetonide on the pharmacokinetic profile of insulin-containing microparticles administered to male Sprague-Dawley rats was evaluated.

Preparation of Insulin-Containing Microparticles, Triamcinolone-Containing Microparticles and Hydrocortisone-Containing Microparticles

Insulin-containing microparticles were prepared as described above. Triamcinolone acetonide-containing microparticles were prepared as described above. Hydrocortisone acetate-containing microparticles were prepared as described above.

Administration of Microparticles

Microparticle administration was as described in Example 1 and treatment groups are summarized in Table 8. A dose of 60 mg of insulin-containing microparticles plus 75 mg of placebo (Group A), 10 mg of 2% w/w triamcinolone acetonide-containing microparticles (Group B) and 15 mg of 2% w/w hydrocortisone-containing microparticles (Group C) was administered to the rats. The rats in this study were immunosuppressed with cyclosporin (Sandimmune, Sandoz; CS) 5 mg/kg only ip daily (except Sundays), for 14 days and three time a week thereafter. Sample collection time points were pre-bleed, 6 hrs, 12 hrs, and days 1, 2, 4, 7, 10, 14, 17, 21, 24, 28, 31, 35, and 38.

TABLE 8
Administration of Insulin-containing Microparticles and Microparticles
Containing a Secondary Agent

| | | | | | Sample Collection |
|---|---|---|---|---|---|
| | | | | | Time points (days) |
| | | INSULIN | | | n = 5 (first 5 in each |
| Group | # Animals | microparticles | Secondary Agent | Treatment | group) |
| A. | 10 | 60 mg | Placebo | 75 mg | pre-bleed, 6 hrs, |
| | | | microparticles | | 12 hrs, day 1, 2, 4, 7, |
| | | | | | 10, 14, 17, 21, 24, |
| | | | | | 28, 31, 35, and 38 |
| B. | 10 | 60 mg | 2% Triamcinolone | 10 mg | same as above |
| | | | acetonide | | |
| | | | microparticles | | |
| C. | 10 | 60 mg | 2% Hydrocortisone | 15 mg | same as above |
| | | | microparticles | | |

Serum Evaluation

To evaluate serum insulin levels, 0.4 mL samples of serum were collected via tail vein on the days specified in Table 8 (four animals per group). After clotting, the samples were centrifuged, aliquoted (3 sets, 54 μL each tube) and frozen (−80° C.). Serum insulin levels were quantitated by ELISA (ALPCO) according to the manufacturer's instructions (Cat. No. 008-10-1132-01).

RNA Analyses:

RNA was extracted from microsphere beds using a Qiagen RNeasy kit as described by the manufacturer. The purified RNA was used to synthesize cDNA using Promega's Reverse Transcriptase kit as described by the manufacturer. Osteopontin cDNA was measured in the samples using real time polymerase chain reaction and osteopontin-specific primers obtained from Oligos Etc., Wilsonville, Oreg. Osteopontin mRNA copy number was normalized to GAPDH mRNA levels.

Results

Serum Insulin Levels

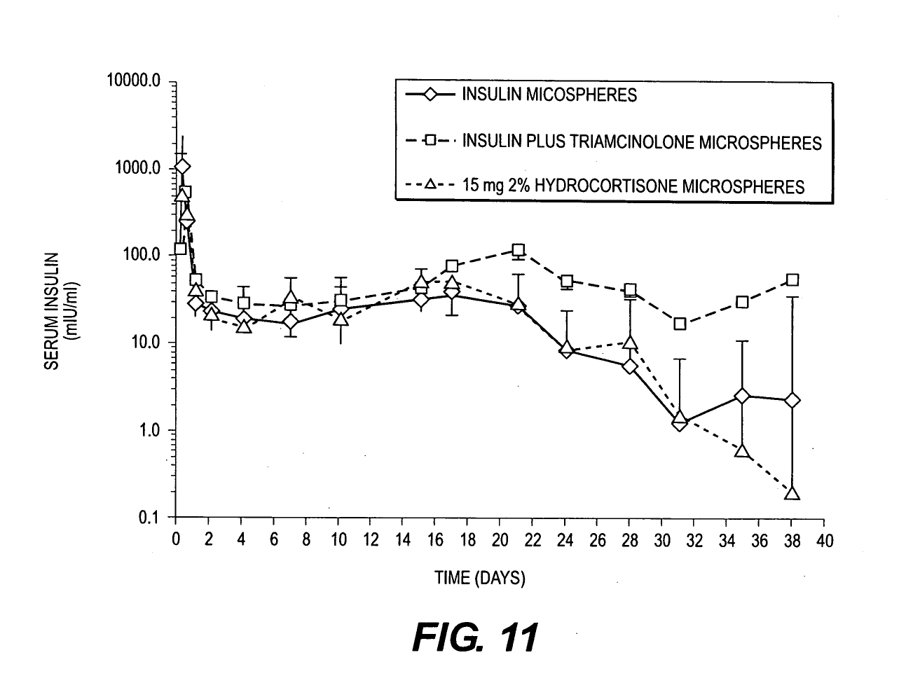

Serum samples were collected as indicated in Table 8 following administration of insulin-containing microparticles co-administered with either placebo microparticles, hydrocortisone- or triamcinolone acetonide-containing microparticles, and tested by ELISA (ALPCO Ultrasensitive Insulin) for serum insulin levels. FIG. 11 shows the pharmacokinetic profile for each group over the course of the study in the form of a graph of serum insulin levels (mIU/mL) in rats administered 60 mg of insulin-containing microparticles plus 75 mg of placebo, 10 mg of 2% w/w triamcinolone acetonide-containing microparticles or 15 mg of 2% w/w hydrocortisone-containing microparticles over time (days). As shown in FIG. 11, there were no significant differences of treated groups compared to controls during the burst phase in serum levels of insulin. The insulin release profile showed a steady release curve in all the groups, with serum levels dropping off by day 2, and increasing slightly until about day 17.

Following the decrease in serum insulin levels post-burst, the highest serum levels occurred at about day 17. At day 17, serum levels in animals administered insulin-containing microparticles plus triamcinolone acetonide-containing microparticles (Group B) were significantly higher (p≦0.05) than control animals administered insulin-containing microparticles plus placebo microparticles (33.3±20.08 mIU/mL), at 79.8±28.5. In addition, after day 17 serum insulin levels only in the Group B animals remained significantly higher than the control group (Group A). The control group had serum levels of 2.5±3.8 mIU/mL by day 35. However, serum levels in rats treated with insulin microparticles co-administered with triamcinolone were significantly higher at 30.5±10.8 mIU/mL at day 31. These results indicate that triamcinolone-containing microparticles increased the sustained release properties of insulin-containing microparticles.

Bioavailability

In terms of bioavailability, the group receiving the triamcinolone microparticles co-administered with insulin microparticles (Group B) had the highest total area under the curve (AUC) at 2045.0±620.3 mIU/mL (Table 5), which was significantly higher than control animals (Group A) at 1021.3±396.7 mIU/mL as shown in Table 9 (p=0.05). Post-burst AUC (days 2-35) were highest in the triamcinolone acetonide treated rats at 1744.8±582.4 mIU/mL compared to 614.6±213.9 mIU/mL in controls, and this difference in post-burst AUC is significant (p=0.05). In addition, the average serum insulin levels between day 2 and 38 were higher in the triamcinolone acetonide treated animals relative to controls (control group 16.9±5.9, triamcinolone group 48.0±16.4) being significantly different from controls (p<0.05). These data indicate that triamcinolone increases the bioavailability of insulin release from microparticles.

TABLE 9
Bioavailability of Insulin-containing Microparticles Co-administered with
Microparticles Containing a Secondary Agent

| | | Steady State | Post-burst AUC | Total | |
|---|---|---|---|---|---|
| Treatment | Cmax | (d2-38) | (d32-2) | AUC | 0-2/total |
| 75 mg Placebo microparticles | 1181.2 ± 1285.8 | 16.9 ± 5.7 | 614.6 ± 213.9 | 1021.3 ± 396.7 | 37.3 ± 16.1 |
| 10 mg 2% Triamcinolone | 632.1 ± 732.4 | 48.0 ± 16.4 | 1744.8 ± 582.4 | 2045.0 ± 620.3 | 14.5 ± 10.0 |
| acetonide microparticles | | | | | |
| 15 mg 2% Hydrocortisone | 733.4 ± 586.4 | 20.9 ± 4.7 | 775.0 ± 172.5 | 1080.6 ± 231.3 | 28.2 ± 8.0 |
| microparticles | | | | | |

Injection Site Analysis:
RT-PCR

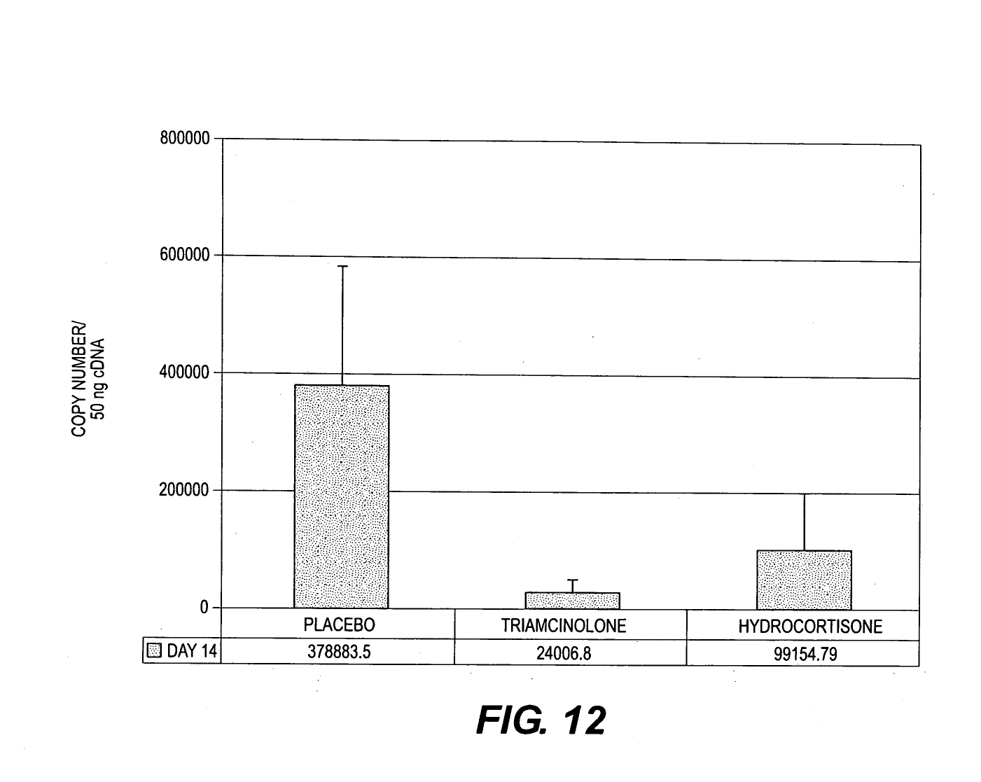

The level of osteopontin mRNA extracted from microsphere beds 14 days post injection was measured by real time reverse transcriptase PCR and osteopontin specific markers obtained from Oliogs Etc. of Wilsonville, Oreg. The results of the real time reverse transcriptase analysis are shown in FIG. 12, which is a histogram of osteopontin mRNA expression levels (copy numbers/50 ng cDNA) in rats administered 60 mg of insulin-containing microparticles plus 75 mg of placebo (Placebo), 10 mg of 2% w/w triamcinolone acetonide-containing microparticles (Triamcinolone) or 15 mg of 2% w/w hydrocortisone-containing microparticles (Hydrocortisone) at day 14 after administration. As shown in FIG. 12, co-injection of triamcinolone acetonide-containing microparticles with insulin-containing microparticles had the most dramatic effects on osteopontin mRNA with levels 93% lower than placebo microsphere controls. Hydrocortisone-containing microparticles suppressed osteopontin mRNA levels by 73% compared to controls.

These results demonstrate that coadministration of triamcinolone acetonide-containing or hydrocortisone-containing microparticles with insulin-containing microparticles decreased inflammation, as assessed by measuring decreased levels of the pro-inflammatory cytokine osteopontin in treated rats.

Immunohistochemistry:

Immunohistochemical analyses of the injection site were also carried out. These studies demonstrated that triamcinolone microparticles co-administered with insulin-containing microparticles dramatically reduced the infiltration of macrophages, monocytes, and T cells to the insulin-containing microparticles at day 14 post-injection. While the hydrocortisone microparticles also reduced inflammatory cell recruitment, their effect was less than the triamcinolone microparticles.

### Example 9

### Effect of Local Delivery of Microparticles Containing a Secondary Agent on the Release of Insulin from Insulin-Containing Microparticles and Cytokines Expression

The effects on the release of insulin from insulin-containing microparticles co-administered to male Sprague-Dawley rats with placebo microparticles, or triamcinolone acetonide- or hydrocortisone-containing microparticles, as well as on the expression of various cytokines at the injection site was determined.

Preparation of Insulin-Containing Microparticles, Triamcinolone-Containing Microparticles and Hydrocortisone-Containing Microparticles

Insulin-containing microparticles were prepared as described above. Triamcinolone acetonide-containing microparticles and hydrocortisone acetate-containing microparticles were prepared as described in Example 8. Placebo microparticles were the same as used in Example 8. The rats used in this study were immunosuppressed using cyclosporin as described in Example 8.

Administration of Microparticles

Microparticle administration, sample collection and analysis were as described in Example 8 and are summarized in Table 10. A dose of 60 mg of insulin-containing microparticles plus 25 mg of placebo (Group A), 10 mg of 2% w/w triamcinolone acetonide-containing microparticles (Group B) or 15 mg of 2% w/w hydrocortisone-containing microparticles (Group C) was administered to the rats. Sample collection time points were pre-bleed, 6 hrs, 12 hrs, and days 1, 2, 4, 7, 14, 21, 28, and 35.

TABLE 10
Administration of Insulin-containing Microparticles and Microparticles
Containing a Secondary Agent

| | | | | | Sample Collection |
|---|---|---|---|---|---|
| | | | | | Time points (days) |
| | | INSULIN | | | n = 5 (first 5 in each |
| Group | # Animals | microparticles | Secondary Agent | Treatment | group) |
| A. | 10 | 60 mg | Placebo | 25 mg | pre-bleed, 6 hrs, |
| | | | microparticles | | 12 hrs, day 1, 2, 4, 7, |
| | | | | | 14, 21, 28 and 35 |
| B. | 10 | 60 mg | 2% triamcinolone | 10 mg | same as above |
| | | | acetonide | | |
| | | | microparticles | | |
| C. | 10 | 60 mg | 2% hydrocortisone | 15 mg | same as above |
| | | | microparticles | | |

Evaluation Parameters
Serum Insulin Levels

To measure serum insulin levels, serum samples (400 μL) were collected via tail vein on the following days relative to microparticle administration: pre-bleed, 1, 2, 4, 7, 10, 14, 17, 21, 28, 31 and 35. After clotting, the samples were prepared for freezing as described in Example 8, and serum insulin levels were quantitated as described in Example 8.

RNA Analyses

RNA was extracted from the microsphere beds using Qiagen RNeasy kit as described by the manufacturer. The purified RNA was used to make cDNA using Promega's Reverse Transcriptase kit as described by the manufacturer. Osteopontin cDNA was measured in the samples using real time polymerase chain reaction and osteopontin-specific primers obtained from Oligos Etc. of Wilsonville, Oreg. Osteopontin mRNA copy number was normalized to GAPDH mRNA levels. Pro-inflammatory chemokine expression was visualized using BioSource's Chemokine Panel A and B PCR kits. Expression of the various chemokines was visualized on a ethidium bromide-containing 2% agarose gel.

Results

Serum Insulin Levels

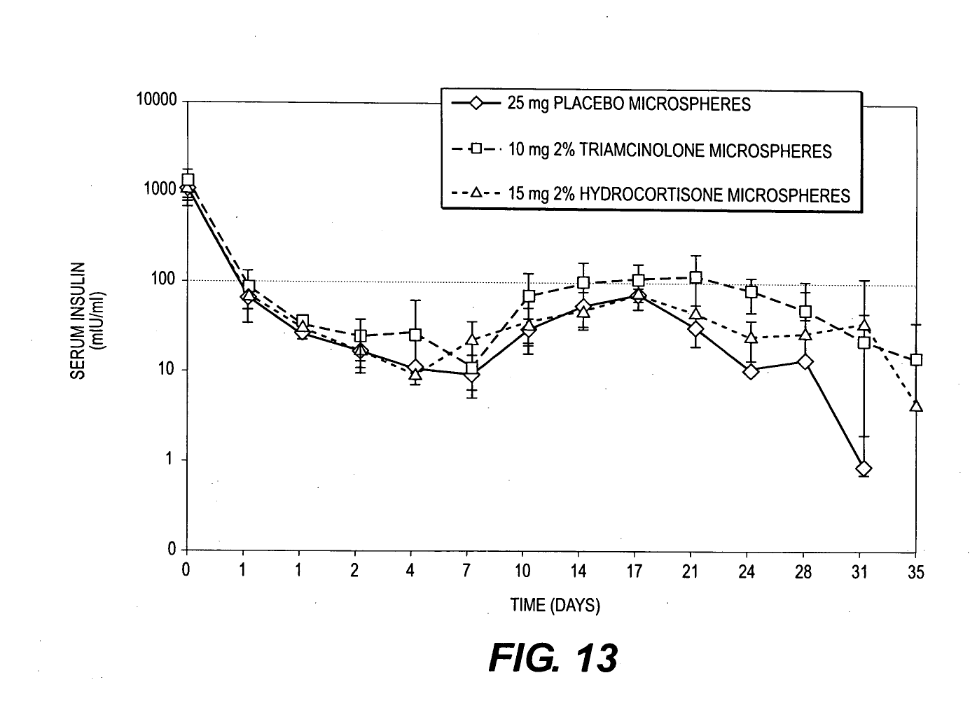

FIG. 13 shows the results of the effects of insulin-containing microparticles co-administered with placebo microparticles, triamcinolone acetonide-containing microparticles or hydrocortisone-containing microparticles on serum insulin levels. As shown in FIG. 13, Group A animals (administered insulin-containing microparticles plus placebo microparticles) demonstrated the shortest pharmacokinetic profile with no detectable serum insulin after 31 days. Group B animals (administered insulin-containing microparticles plus triamcinolone acetonide-containing microparticles) demonstrated the highest levels of insulin in the serum from day 2-until the end of the study (day 35) at which time insulin was still measurable in the serum. The presence of a secondary agent also increased the postburst AUC relative to the placebo-treated group by 149.6% and 38.07% for groups administered insulin-containing microparticles plus triamcinolone acetonide- and hydrocortisone-containing microparticles, respectively.

These results indicate that triamcinolone and hydrocortisone prolonged the period of sustained release of insulin from insulin-containing microparticles in comparison to release from insulin-containing microparticles administered alone.

Pro-Inflammatory Cytokine Expression

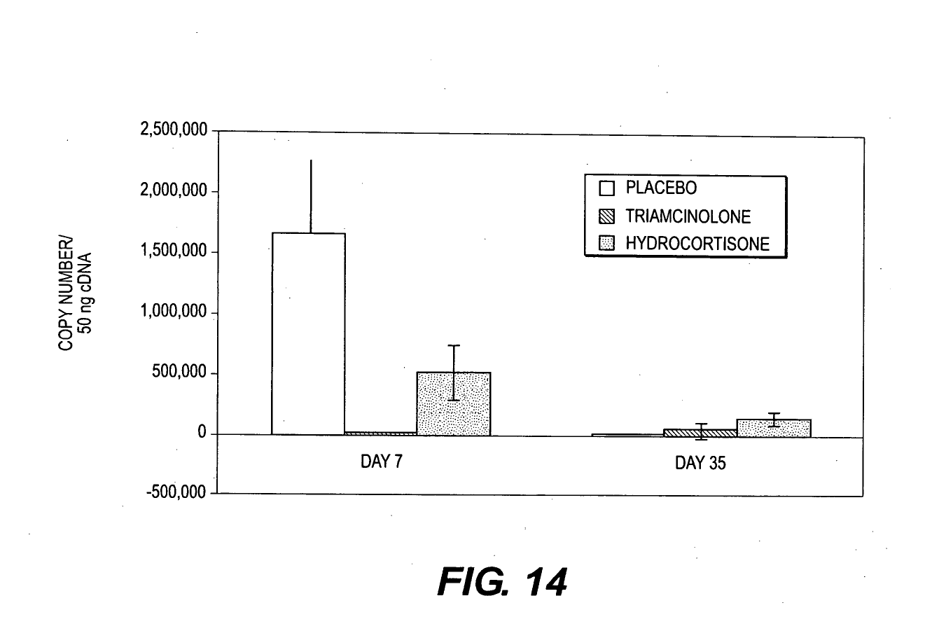

Analysis of mRNA levels of several pro-inflammatory cytokines extracted from microsphere injections sites by reverse transcriptase PCR, demonstrated the presence of mRNA for a number of pro-inflammatory chemotactic factors including osteopontin, RANTES, MIP-1α, MIP-1β, MCP-1, and MIP-2. Osteopontin mRNA levels were quantitated, and found to be highest in the placebo group at day 7 post-injection, as shown in FIG. 14, which is a histogram of osteopontin mRNA expression levels (copy numbers/50 ng cDNA) in rats administered 60 mg of insulin-containing microparticles plus 25 mg of placebo (Placebo), 10 mg of 2% w/w triamcinolone acetonide-containing microparticles (Triamcinolone) or 15 mg of 2% w/w hydrocortisone-containing microparticles (Hydrocortisone) at days 7 and 35 after administration. The animal group administered insulin-containing microparticles plus triamcinolone acetonide-containing microparticles had 200 times less osteopontin mRNA transcript than the placebo, and the groups administered insulin-containing microparticles plus hydrocortisone-containing microparticles displayed approximately one-half as much osteopontin transcript than the placebo group. At day 35 the level of osteopontin mRNA was low in all groups.

### Example 10

### Effects of Local Delivery of Secondary Agent-Containing Microparticles on the Release of Exendin-4 from Exendin-Containing Microparticles

The effects on the pharmacokinetic profile of exendin release following administration of exendin-containing microparticles co-administered to male Sprague-Dawley rats with placebo microparticles, or triamcinolone-containing microparticles as determined.

Preparation of Exendin-Containing Microparticles and Triamcinolone-Containing Microparticles

Exendin-containing microparticles were prepared as described above. Triamcinolone acetonide-containing microparticles were prepared as described above. Placebo microparticles were prepared as described above.

Administration of Microparticles

Microparticle administration was as described in Example 1 and treatment groups are summarized in Table 11. A dose of 120 mg of exendin-containing microparticles designated IF-1 plus 30 mg of placebo (Group A) or 10 mg of 2% w/w triamcinolone-containing microparticles (Group B) was administered to the rats. A dose of 40 mg of exendin-containing microparticles designated SF-2 plus 30 mg of placebo (Group C) or 10 mg of 2% w/w triamcinolone-containing microparticles (Group D) was also administered to the rats. Sample collection time points were pre-bleed, 2 hrs, 6 hrs, 10 hrs, and days 1, 2, 4, 7, 10, 14, 17, 21, 24, 29, 32, 36 and 39.

TABLE 11
Administration of Exendin-containing Microparticles and Microparticles
Containing a Secondary Agent

| | | EXENDIN | | |
|---|---|---|---|---|
| Group | # Animals | microparticles | Secondary Agent | Treatment |
| A. | 4 | 120 mg | Placebo | 30 mg |
| | | IF-1 | microparticles | |
| B. | 4 | 120 mg | 2% Triamcinolone | 10 mg |
| | | IF-1 | microparticles | |
| C. | 4 | 40 mg | Placebo | 30 mg |
| | | SF-2 | microparticles | |
| D. | 4 | 40 mg | 2% Triamcinolone | 10 mg |
| | | SF-2 | microparticles | |

Plasma Evaluation

To evaluate plasma exendin levels, 0.25 mL samples of plasma were collected via tail vein on days 0 and 1, and 0.4 mL samples were collected on the remaining days specified in Table 11 (four animals per group). The samples, were centrifuged and the plasma fraction frozen (−80° C.). Plasma exendin levels were quantitated by IRMA describe below

In Vivo Release-IRMA

The method for quantifying exendin-4 in plasma is a sandwich immunoassay, with the analyte captured by a solid phase monoclonal antibody EXE4:2-8.4 and detected by the radioiodinated monoclonal antibody GLP-1:3-3. Counts bound are quantitated from a standard calibration curve. This assay is specific for exendin-4 and does not detect exendin-4 (3-39) a major metabolite or GLP-1. A typical standard curve range is 30 pg/mL to 2000 pg/mL depending on the age of the tracer antibody.

Results

Plasma Exendin-4 Levels

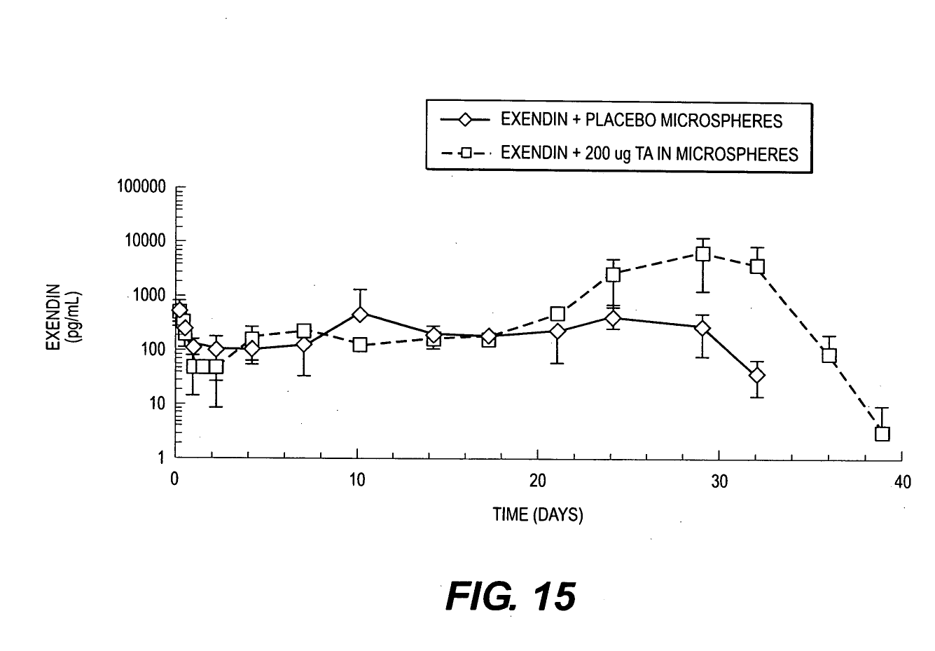

FIG. 15 shows the results of the effects of exendin-4-containing microparticles co-administered with placebo microparticles and triamcinolone acetonide-containing microparticles on plasma exendin levels in the form of a graph of exendin plasma levels (pg/mL) versus time (days) post injection. As shown in FIG. 15, the pharmacokinetic profile for Group B (Lot 02-002-82 and triamcinolone) was improved over controls (Group A). Specifically, enhanced bioavailability was observed for the triamcinolone acetonide treated group (Group B) in that plasma levels on day 32 remained detectable while this was the last day detectable for the control group. It is noted that plasma levels were still detectable at day 39 for Group B, showing a substantial increase in the duration of release of exendin when coadministered with triamcinolone acetonide-containing microparticles. Cₐᵥₑ levels, Cₘₐₓ and AUC were also desirably modulated as a result of coadministration of triamcinolone acetonide-containing microparticles with the exendin-containing microparticles.

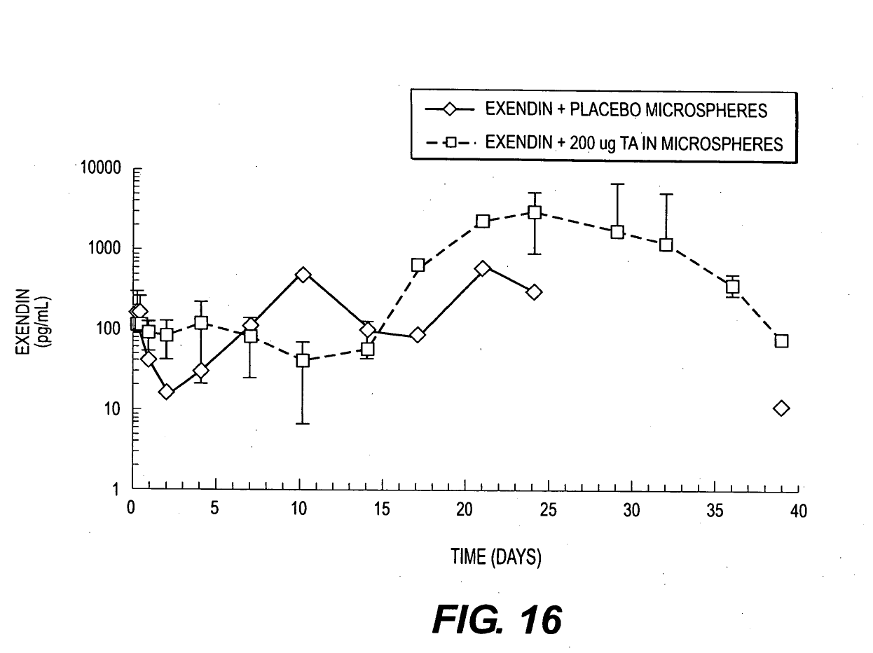

FIG. 16 shows the results of the effects of exendin-containing microparticles co-administered with placebo microparticles and triamcinolone acetonide-containing microparticles on serum exendin levels in the form of a graph of exendin serum levels (pg/mL) versus time (days) post injection. As shown in FIG. 16, the pharmacokinetic profile for Group D (Lot 01-011-49C and triamcinolone acetonide) was improved over controls (Group C). Specifically, enhanced bioavailability was observed for the triamcinolone treated group (Group D) in that plasma levels were still detectable at day 39 showing a substantial increase in the duration of release of exendin when coadministered with triamcinolone acetonide-containing microparticles in comparison to controls (Group C) which were not detectable after day 24. Cₐᵥₑ levels, Cₘₐₓ and AUC were also desirably modulated as a result of coadministration of triamcinolone acetonide-containing microparticles with the exendin-containing microparticles.

While this invention has been particularly shown and described with references to preferred embodiments thereof, it will be understood by those skilled in the art that various changes in form and details may be made therein without departing from the scope of the invention encompassed by the appended claims.

We claim:

1. A method for the sustained release in vivo of a peptide, comprising:
administering to a subject-a sustained-release composition comprising a poly(lactide-co-glycolide) polymer having the peptide and a corticosteroid incorporated therein, wherein the peptide is released for a period of at least about two weeks, wherein the corticosteroid is present in an amount sufficient to modify the release profile of the peptide from the polymer and provide increased bioavailability of the peptide.

2. A composition for the sustained release of a peptide, comprising:
a poly(lactide-co-glycolide) polymer having the peptide and a corticosteroid incorporated therein, wherein the peptide is released for a period of at least about two weeks, wherein the corticosteroid is present in an amount sufficient to modify the release profile of the peptide from the polymer and provide increased bioavailability of the peptide.
